(19) United States
(12) Patent Application Publication
(10) Pub. No.: US 2009/0024204 A1
(43) Pub. Date: Jan. 22, 2009

(54) EXPANDABLE SUPPORT DEVICE AND METHOD OF USE

(75) Inventors: E. Skott GREENHALGH, Lower Gwynedd, PA (US); John-Paul ROMANO, Chalfont, PA (US)

Correspondence Address: LEVINE BAGADE HAN LLP, 2483 EAST BAYSHORE ROAD, SUITE 100, PALO ALTO, CA, 94303, US

(73) Assignee: Stout Medical Group, L.P., Perkasie, PA (US)

(21) Appl. No.: 12/098,297

(22) Filed: Apr. 4, 2008

Related U.S. Application Data
(63) Continuation-in-part of application No. PCT/US2006/038920, filed on Oct. 4, 2006.
(60) Provisional application No. 60/723,309, filed on Oct. 4, 2005.
(60) Provisional application No. 60/735,718, filed on Nov. 11, 2005.

(51) Int. Cl. A61F 2/06; A61B 19/00

(52) U.S. Cl. 623/1.15; 623/1.37; 623/1.39; 128/898

(57) ABSTRACT

A closable-tip fracture stent for tissue repair is disclosed. The device can be used to repair hard or soft tissue, such as bone or vertebral discs. A method of repairing tissue is also disclosed. The device comprises a flexible or semi-rigid wall, defining an interior cavity, and one or more closable tips to close the hollow cavity. A delivery tool is also provided for removably carrying the orthopedic device to the treatment site.

### CROSS-REFERENCE TO RELATED APPLICATIONS

This application is a continuation-in-part of PCT International Application No. PCT/US2006/038920, filed Oct. 4, 2006 which claims the benefit of U.S. Provisional Application Nos. 60/723,309, filed Oct. 4, 2005, and 60/735,718, filed Nov. 11, 2005 which are herein incorporated by reference in their entireties.

### BACKGROUND OF THE INVENTION

### 1. Field of the Invention

This invention relates to devices for providing support for biological tissue, for example to repair bone fractures, for example damaged vertebra, and methods of using the same.

This invention relates to devices for providing support for biological tissue, for example to repair spinal compression fractures, and methods of using the same.

Vertebroplasty is an image-guided, minimally invasive, nonsurgical therapy used to strengthen a broken vertebra that has been weakened by disease, such as osteoporosis or cancer. Vertebroplasty is often used to treat compression fractures, such as those caused by osteoporosis, cancer, or stress.

Vertebroplasty is often performed on patients too elderly or frail to tolerate open spinal surgery, or with bones too weak for surgical spinal repair. Patients with vertebral damage due to a malignant tumor may sometimes benefit from vertebroplasty. The procedure can also be used in younger patients whose osteoporosis is caused by long-term steroid treatment or a metabolic disorder.

Vertebroplasty can increase the patient's functional abilities, allow a return to the previous level of activity, and prevent further vertebral collapse. Vertebroplasty attempts to also alleviate the pain caused by a compression fracture.

Vertebroplasty is often accomplished by injecting an orthopedic cement mixture through a needle into the fractured bone. The cement mixture can leak from the bone, potentially entering a dangerous location such as the spinal canal. The cement mixture, which is naturally viscous, is difficult to inject through small diameter needles, and thus many practitioners choose to “thin out” the cement mixture to improve cement injection, which ultimately exacerbates the leakage problems. The flow of the cement liquid also naturally follows the path of least resistance once it enters the bone—naturally along the cracks formed during the compression fracture. This further exacerbates the leakage.

The mixture also fills or substantially fills the cavity of the compression fracture and is limited to certain chemical composition, thereby limiting the amount of otherwise beneficial compounds that can be added to the fracture zone to improve healing. Further, a balloon must first be inserted in the compression fracture and the vertebra must be expanded before the cement is injected into the newly formed space.

A vertebroplasty device and method that eliminates or reduces the risks and complexity of the existing art is desired. A vertebroplasty device and method that is not based on injecting a liquid directly into the compression fracture zone is desired.

### SUMMARY OF THE INVENTION

A fracture stent is disclosed. The fracture stent can be hollow. The fracture stent can have a tip that can remain open during insertion into the fracture repair site. The tip can become closed in response to the being forced against the terminal end of the prepared fracture repair site. The tip can be manually closed through external closure means once it has been inserted to the necessary place. Any biological material that is in the repair site prior to the insertion of the closable tip fracture stent can slide into the hollow interior of the fracture stent, for example, instead of being displaced or forced out. The fracture stent can produce a less traumatic procedure for the patient.

The fracture stent can have a closable tip. The fracture stent can have a porous wall. Biologically active material in the repair site prior to the insertion of the fracture stent, such as blood, bone marrow, or other tissue, can remain within the repair site. The porosity of the wall can allow the biological material in the repair site that subsequently enters the hollow cavity within the fracture stent to interact with the surrounding bone 142 of the repair site. The biologically active material in the repair site can encourage the natural healing process and expedite the repair of the fracture.

The fracture stent with can tightly fit in the repair site. The fracture stent does not require that the biological material that is present within the repair site prior to the insertion of the repair stent be removed or forced from the repair site. The open tip can force the biological material from the path of entry, for example, to slide to the center of the fracture stent. The fracture stent can be sized to have a very close fit with the inner wall of the repair site. No gap is required to allow the escape of any biological material in the repair site. The closable open tip can be configured to not seal the stent until the stent has reached the desired location in the repair site.

The tight fit of the fracture stent can result in a more stable and secure repair. The tight fit can allow the patient to resume a normal range of activities earlier.

### BRIEF DESCRIPTION OF THE DRAWINGS

FIGS. 1 and 2 are side views of various embodiments of the closable-tip fracture stent.

FIG. 3 is a front view of the embodiment of the closable-tip fracture stent of FIG. 2.

FIG. 4 is a side view of an embodiment of the closable-tip fracture stent.

FIGS. 5 through 9 are front views of various embodiments of the closable-tip fracture stent.

FIG. 7 is a bottom view of an embodiment of the closable-tip fracture stent.

FIG. 8 is a side view of an embodiment of the closable-tip fracture stent.

FIG. 9 is a top view of an embodiment of the closable-tip fracture stent.

FIG. 10 is a bottom view of an embodiment of the closable-tip fracture stent.

FIG. 11 is a side view of an embodiment of the closable-tip fracture stent.

FIG. 12 is a side view of an embodiment of the closable-tip fracture stent.

FIG. 13 is a front transparent view of an embodiment of the closable-tip fracture stent.

FIGS. 14 and 15 are side views of various embodiments of the closable-tip fracture stent.

FIG. 16 is a side view of an embodiment of a deployment tool.

FIG. 17 is a side view of an embodiment of the closable-tip fracture stent with the deployment tool of FIG. 16.

FIG. 18 is a bottom view of the embodiment of the closable-tip fracture stent with the deployment tool of FIG. 16.

FIG. 19 is a side view of an embodiment of the closable-tip fracture stent with the deployment tool of FIG. 16.

FIG. 20 is a bottom view of an embodiment of the closable-tip fracture stent with the deployment tool of FIG. 16.

FIGS. 21 through 25 are side views of various embodiments of the closable-tip fracture stent.

FIG. 26 is a cut-away side view of an embodiment of the closable-tip fracture stent.

FIG. 27 is a cut-away close-up side view of an embodiment of the closable-tip fracture stent.

FIG. 28 is a side view of an embodiment of the closable-tip fracture stent.

FIG. 29 is a side view of an embodiment of a deployment tool for the closable-tip fracture stent.

FIGS. 30 and 31 are cut-away side views of a method of using the closable-tip fracture stent.

FIGS. 32 and 33 illustrate side views of elements of an embodiment of the closable-tip fracture stent.

FIG. 34 is a side view of an embodiment of the closable-tip fracture stent.

FIGS. 35 through 37 are side views of various embodiments of the closable-tip fracture stent.

FIG. 38 is a cut-away detail view of a part of an embodiment of the closable-tip fracture stent.

FIGS. 39 through 41 are side views of various embodiments of a deployment tool for the closable-tip fracture stent.

FIG. 42 illustrates an isometric rear-facing view of an embodiment of the closable-tip fracture repair stent.

FIG. 43 illustrates a front view of the embodiment of the closable-tip fracture repair stent of FIG. 42.

FIG. 44 illustrates a rear view of the embodiment of the closable-tip fracture repair stent of FIG. 42.

FIG. 45 illustrates a side view of the embodiment of the closable-tip fracture repair stent of FIG. 42.

FIGS. 46 through 49 illustrate cut-away side views for methods of using various embodiments of the closable-tip fracture stent.

FIG. 50 illustrates a cut-away side view of a method of using an embodiment of the closable-tip fracture stent.

FIG. 51 illustrates a cut-away detail side view of a method of using an embodiment of the closable-tip fracture stent.

FIGS. 52 through 58 illustrate cut-away side views of various methods for deploying various embodiments of the closable-tip fracture stent into a damage site.

FIGS. 59 through 61 illustrate an embodiment of a method for accessing a damage site in the vertebra.

FIGS. 62 and 63 illustrate a cut-away side view of a damage site in the vertebra.

FIGS. 64 and 65 illustrate a method for deploying various embodiments of the closable-tip fracture stent to repair a damage site in the vertebra.

FIGS. 66 through 74 illustrate various methods for deploying various embodiments of the closable-tip fracture repair stent into damage sites in the vertebra.

FIG. 75 illustrates a side cutaway view of a method for using an embodiment of the closable-tip fracture stent to repair a damage site in the vertebral column.

FIG. 76 illustrates a side cutaway view of a fracture stent deployed in a damage site in a vertebra.

FIG. 77a illustrates a variation of the stent with a covering.

FIG. 77b illustrates a variation of cross-section A-A of FIG. 77a.

FIGS. 78 and 79a illustrate variations of the stent with a covering.

FIGS. 79b and 79c illustrate variations of cross-section B-B of FIG. 79a.

FIGS. 80 through 83 illustrate variations of the stent with a covering.

FIGS. 84 and 86 illustrate a variation of a method for using the cover and the stent.

### DETAILED DESCRIPTION

An expandable support device, such as for implantable orthopedic use, is disclosed. The device comprises a wall, defining an interior cavity, and can have one, two or more closable ends. Delivery devices are also provided for expandably and/or closably deploying the orthopedic device to the treatment site.

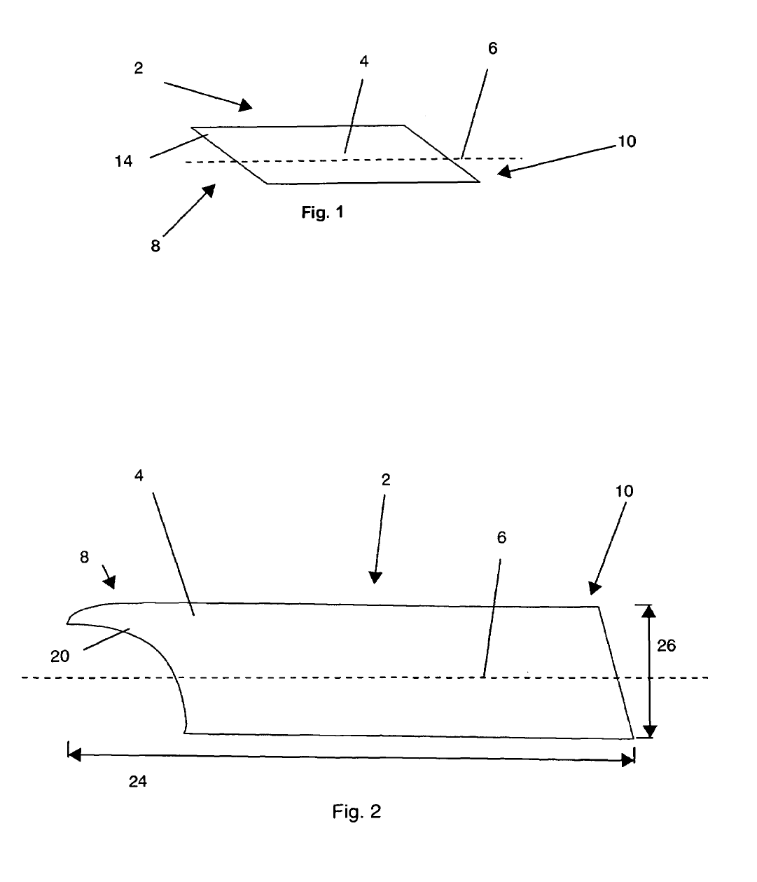

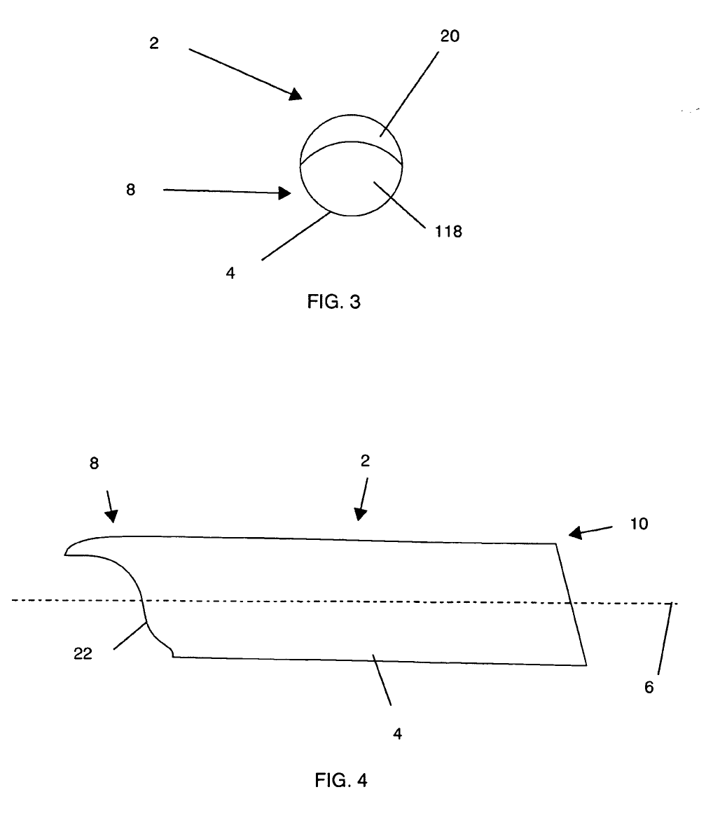

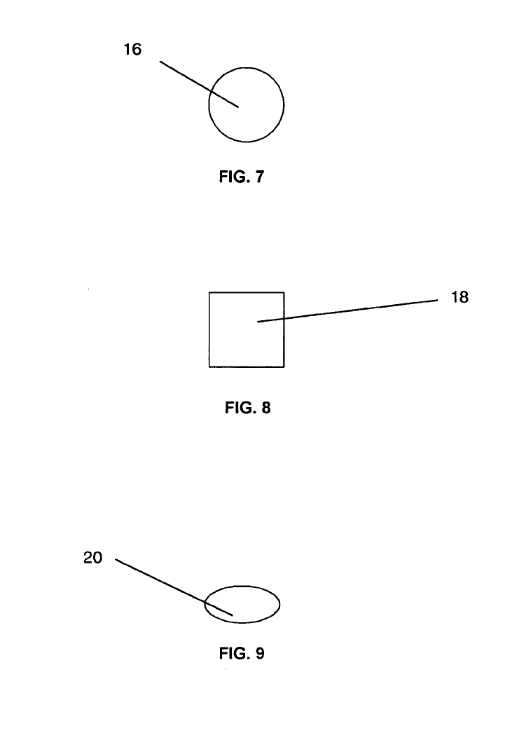

FIGS. 1 through 15 illustrate variations of the expandable support device, such as a closable-tip fracture stent 2. The stent 2 can be implanted in a bone, such as a compression fracture in a vertebra, or in soft tissue, such as a herniated intervertebral disc. The closable-tip fracture stent 2 can be biocompatible. The closable-tip fracture stent 2 can have any configuration, and be used for the methods described herein.

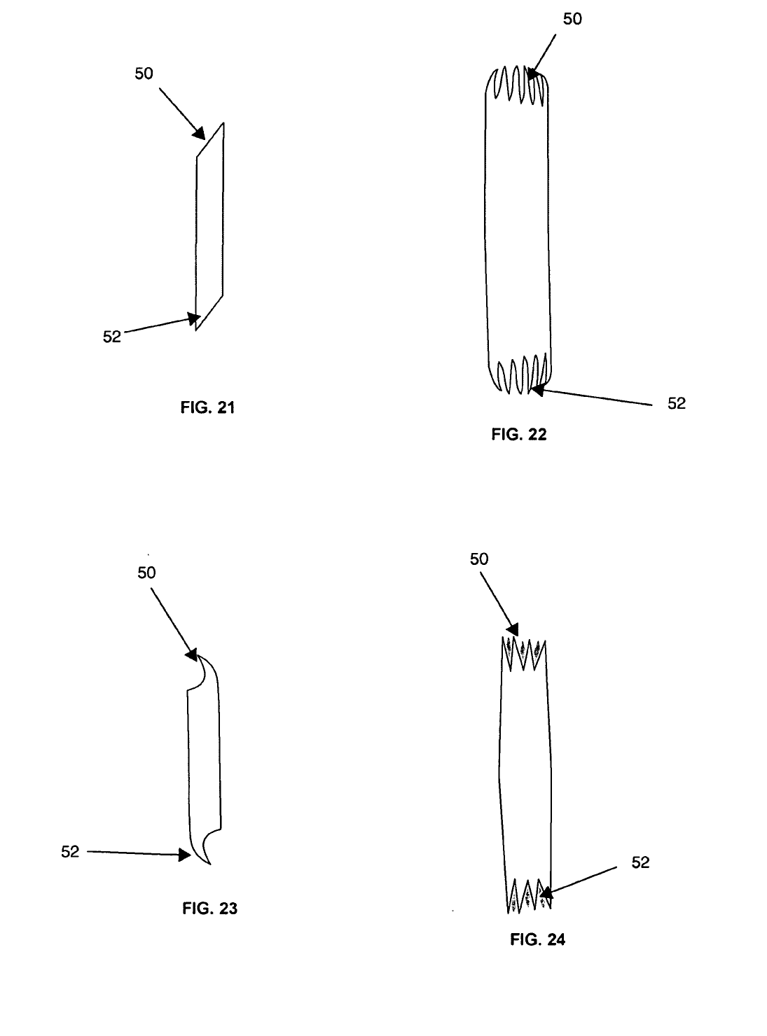

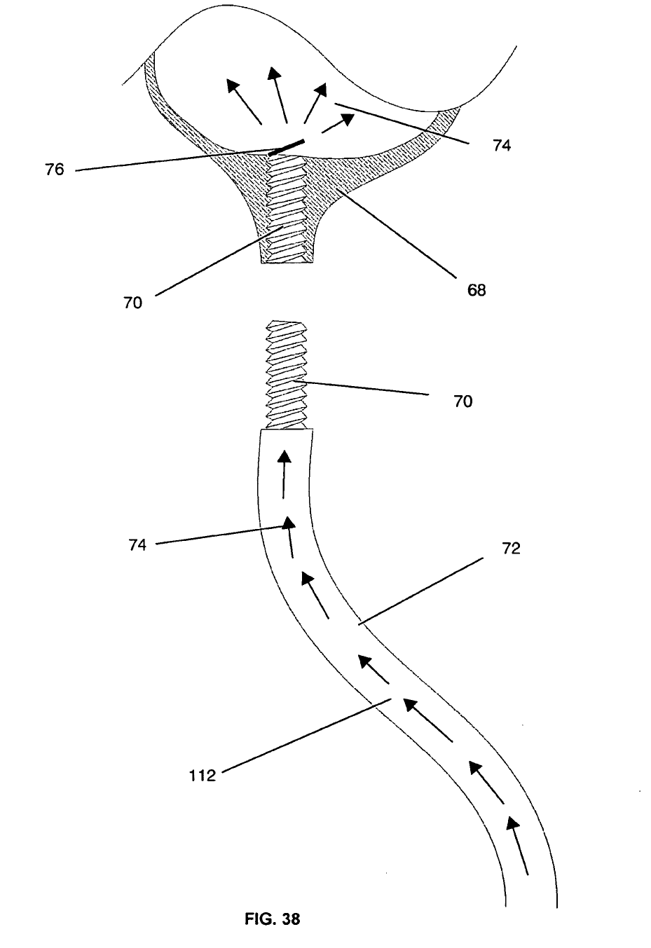

The closable-tip fracture stent 2 can have a wall 4. The wall 4 can define an internal hollow cavity. The closable-tip fracture stent 2 can have a longitudinal axis 6 oriented along the center of the hollow cavity. The closable-tip fracture stent 2 can have a leading end 8 and a trailing end 10. The leading end 8 or trailing end 10, or both ends, can have a tip, which tip can be deformable upon itself in response to force along the longitudinal axis 6. FIGS. 21 through 28, 32 and 34 through 37 illustrate examples of embodiments of the closable tip fracture stent 2 with both deformable leading 8 and trailing 10 ends.

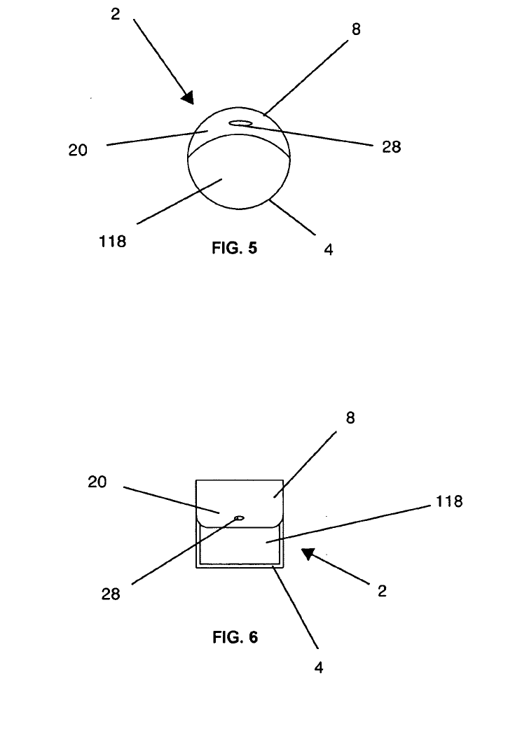

In cross-section the wall 4 can define any hollow shape around the internal cavity, for example, a rectangle, circle, or ellipse. FIGS. 5 through 9 illustrate that the closable-tip fracture stent 2 can have a circular 14, rectangular 16, or elliptical 18 cross-section. The closable-tip fracture stent 2 can also have a combination of shapes of cross-sections along its length.

As illustrated in FIG. 1, the tip can be flat and angled 12 with respect to the longitudinal axis 6. As illustrated in FIGS. 2 and 4, the tip can be curved. Viewed from the side, the profile of a curved tip can be concave, convex, or a combination thereof. FIG. 2 illustrates that the tip can define a curve that is concave 20 with respect to the longitudinal axis 6. FIG. 4 illustrates that the tip can define a curve that is bowed 22 to be both concave and convex with respect to the longitudinal axis 6. The tip can be bowed 22 to define a combination of corresponding convex and concave curves that uniformly meet to substantially close the leading end 8, when the tip is bent down in deployment.

The closable-tip fracture stent 2 can be completely or partially coated with agents and/or matrices as described herein.

The tip of the leading end 8 can be sharpened. The tip of the leading end 8 can be used to help move tissue aside during implantation and deployment. The leading end 8 can be self-penetrating.

As illustrated in FIG. 2, when in a non-deployed configuration, the closable-tip fracture stent 2 can have an open length 24 and an open height 26. The open length 24 can be from about 0.318 cm (0.125 in.) to about 10 cm (4 in.), for example about 3.8 cm (1.5 in). The open height 26 can be from about 0.1 cm (0.05 in.) to about 3 cm (1 in.), for example about 0.8 cm (0.3 in.).

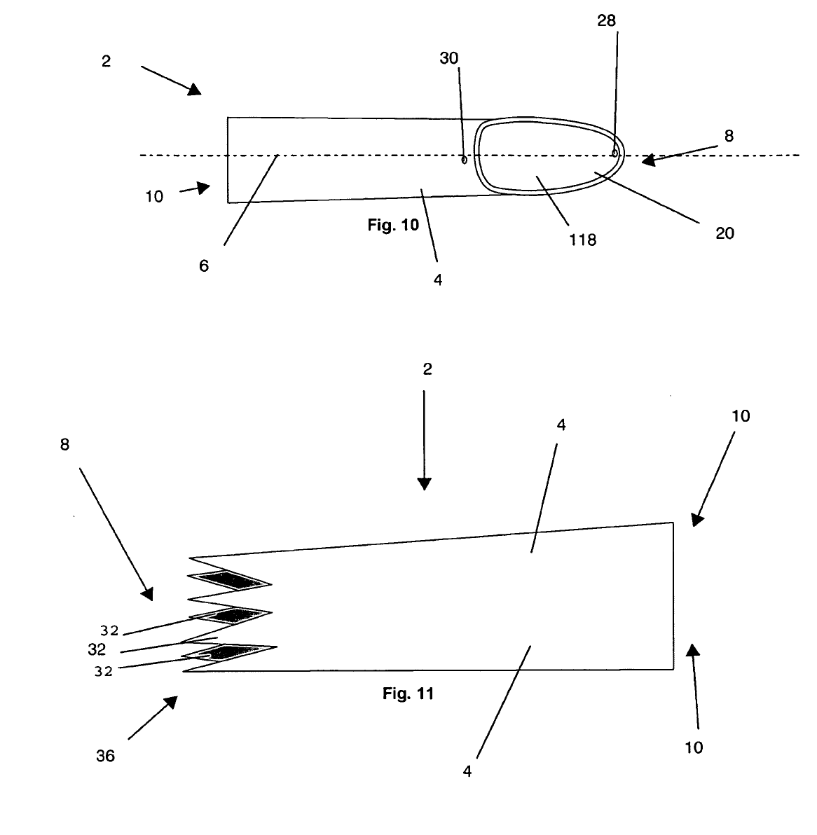

FIGS. 5, 6 and 10 illustrate that the tip can have a first draw eyelet 28 through the tip of its leading 8, or distal 10, end. FIG. 10 illustrates that the closable-tip fracture stent 2 can have a second draw eyelet 30 through its wall 4, located across the hollow opening opposite from the first draw eyelet 28 on the bottom of the leading end 8.

FIGS. 11 and 14 illustrate that the closable-tip fracture stent 2 can have a crowned tip 36 with a plurality of tapered crown points 32. The closable-tip fracture stent 2 can have as few as one crown point or as many as 50 crown points, for example between two and 20 crown points, more narrowly between two and twelve crown points. FIG. 11 illustrates that the closable-tip fracture stent 2 can have about seven crown points.

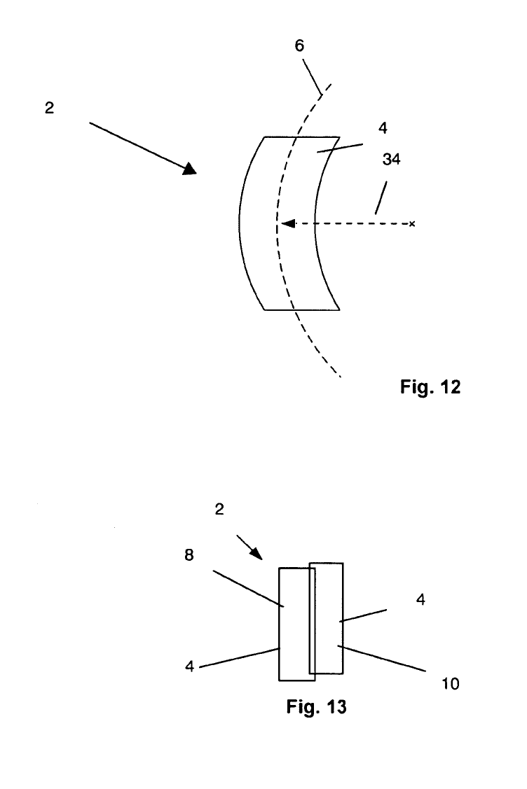

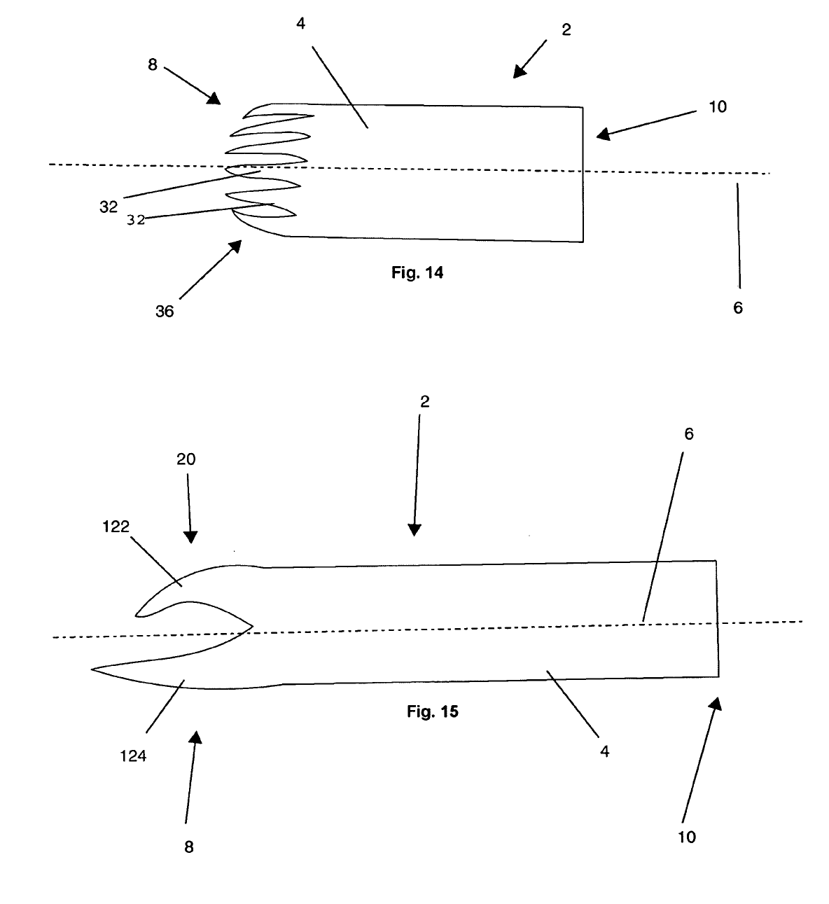

FIGS. 12 and 13 illustrate the closable-tip fracture stent 2 that can have a radius of curvature 34 along the longitudinal axis 6. The radius of curvature 34 can be from about 1 mm (0.04 in.) to about 250 mm (10 in.), for example about 50 mm (2 in.). (The closable-tip fracture stent 2 is shown in FIGS. 12 and 13 without a tip 20 for illustrative purposes.)

FIG. 15 illustrates that the crown points 32 can differ in length on the same closable-tip fracture stent 2. Crown points 32 of differing lengths can be designed to deform over each other upon deployment to substantially close the leading end 8 of the closable-tip fracture stent 2.

The closable-tip fracture stents 2 can have textured and/or porous surfaces for example, to increase friction against bone surfaces, and/or promote tissue ingrowth and/or to allow cements, treatments, preparations, or other fill materials to leak out of the stent into contact with the surrounding bone 142 of the repair site. The closable-tip fracture stents 2 can be coated with a bone growth factor, such as a calcium base.

The outer and/or inner surfaces of the wall 4 can be configured to increase friction with the damage repair site, or be capable of an interference fit with another object, such as another closable-tip fracture stent 2. The configurations to increase friction or be capable of an interference fit include teeth, perforations, knurling, coating, barbs, or combinations thereof. Other configurations to increase friction with the damage repair site can include the use of a shell of interlocking filament or wire mesh. FIG. 13 illustrates an example of an embodiment of a closable tip fracture stent 2 with wire mesh deformable leading 8 and trailing ends 10 to increase friction.

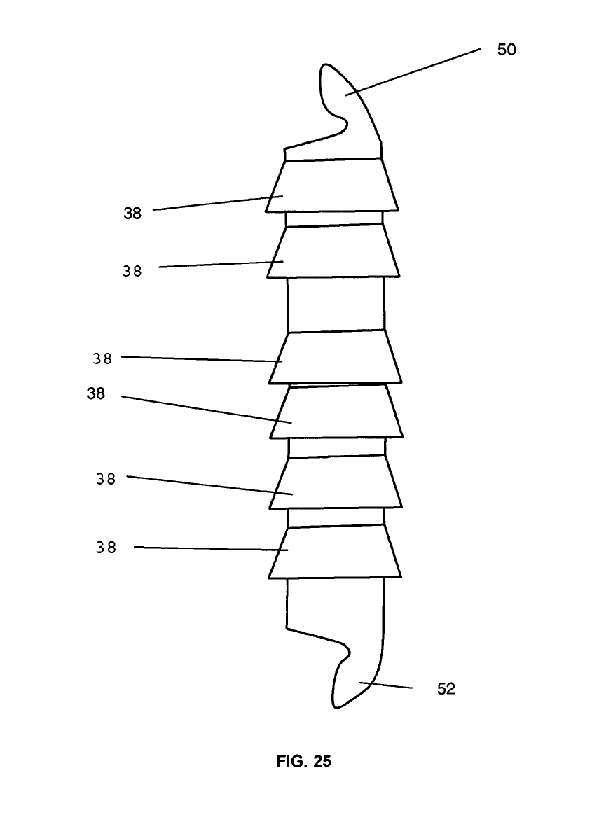

FIG. 25 illustrates an embodiment of the closable tip fracture stent 2 with barbs 38 disposed around its external surface to increase friction.

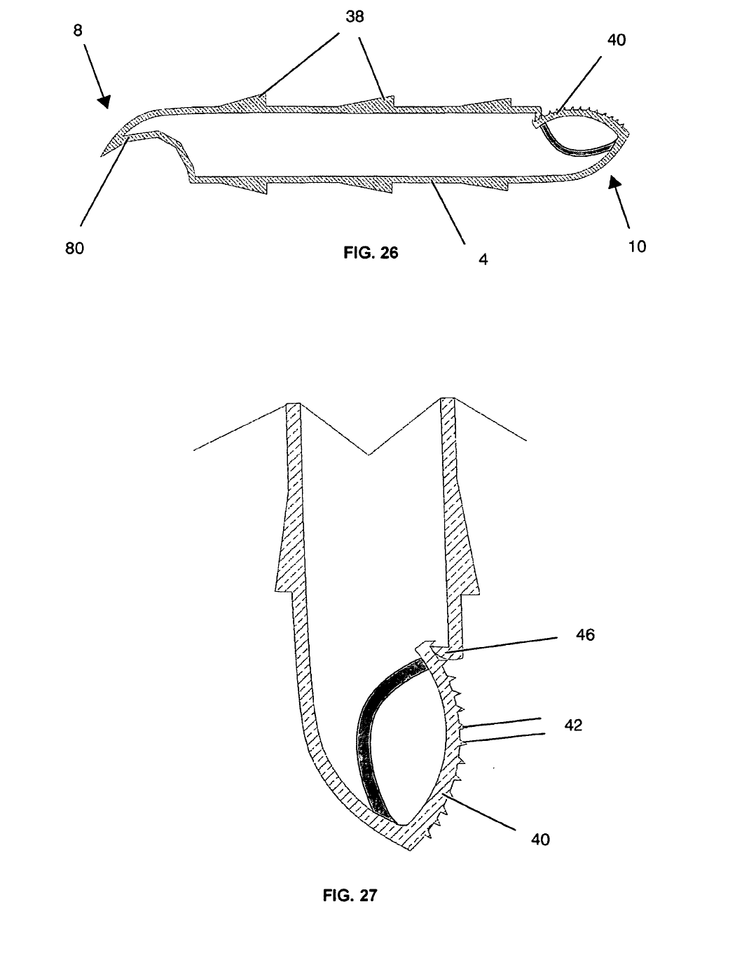

FIGS. 26 and 27 illustrate that the closable tip fracture stent 2 can have a ratchet closing mechanism, for example as illustrated in FIG. 26, on the trailing end 10 or leading end 8, or both, of the closable tip fracture stent 2. As illustrated in FIG. 27, the ratchet closing mechanism can have a semi rigid ratchet strip 40 having ratchet teeth 42 disposed thereon. The ratchet teeth 42 can engage a ratchet catch 46 as illustrated bad FIG. 27. The ratchet catch 46 can allow the ratchet teeth 42 to pass in one direction only, for example, to allow the closable tip fracture stent 2 end tip to be permanently closed, for example by use of a deployment tool. 48

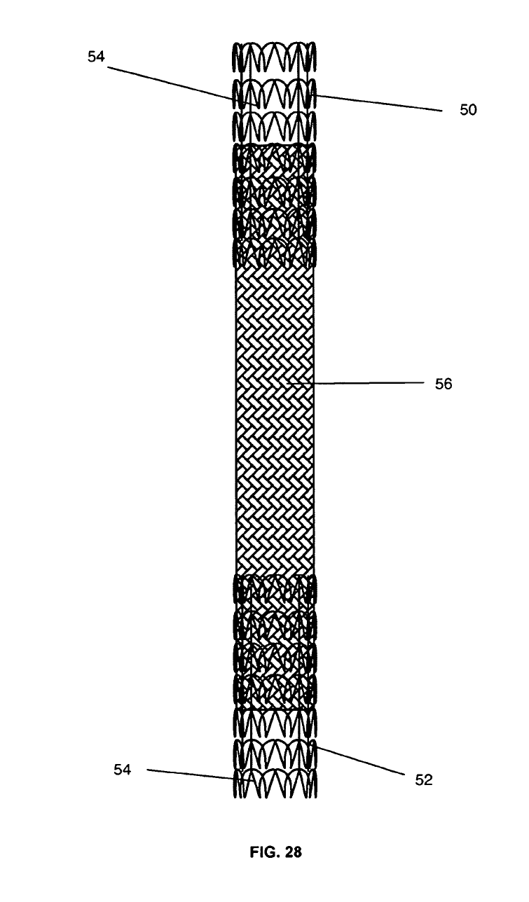

FIGS. 25 and 26 illustrate examples of embodiments of the closable tip fracture stent 2 that have texturization on their outer walls to increase friction. FIG. 28 illustrates an example of an embodiment of a closable tip fracture stent 2 with both a closable leading 50 and trailing end 52. The closable tip fracture stent 2 illustrated in FIG. 28 also shows that wire mesh or interlocking filament elements can be used for the closable tip elements of the stent. As illustrated by FIG. 28 wire mesh closable tip 54 elements can be designed to increase friction. FIG. 28 also illustrates an example of an embodiment of the closable tip fracture stent 2 with a texturized outer surface 56 to increase friction.

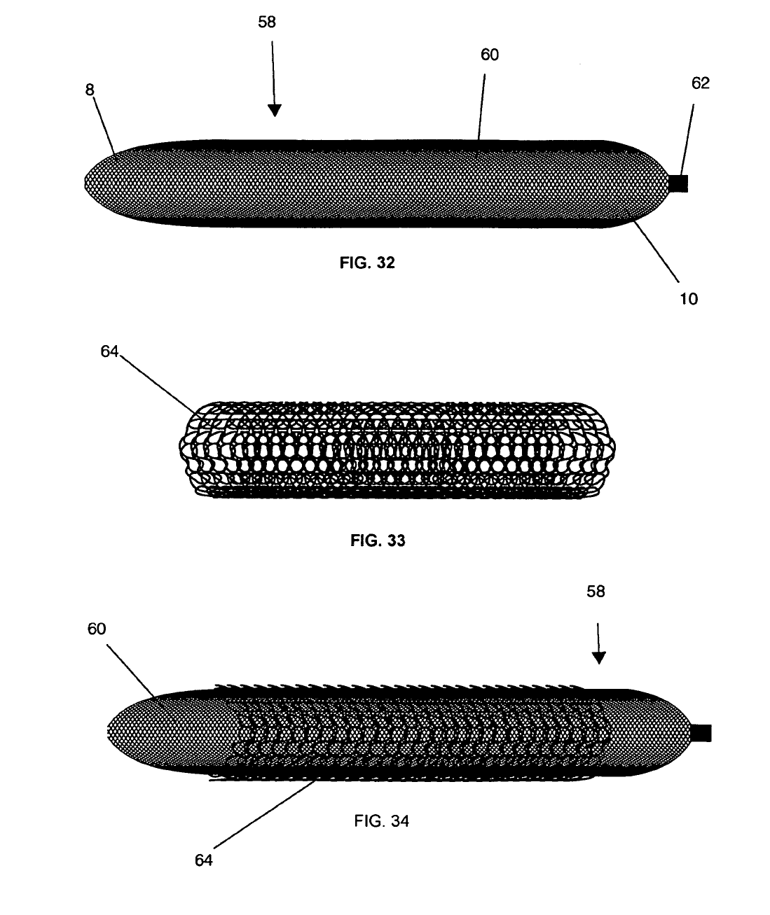

FIG. 32 illustrates an embodiment of a closable tip repair stent 58 with a wall 4 made from woven interlocking filament 60. This design can increase friction with the damage repair site 57. FIG. 32 also illustrates that a closable tip repair stent 58 with a wall 4 made from woven interlocking filament 60 can have a insertion/fill port 62 on its trailing end 10 to engage with an insertion/fill tool 86, for example to maneuver the fracture stent into position in the repair site 57 and fill the fracture stent with a desired fill material 74.

FIG. 33 illustrates an example of an embodiment of a wire mesh external shell 64 that can be used in conjunction with the closable tip fracture stent 2 to increase friction with the damage repair site 57. FIG. 34 illustrates the wire mesh shell 64 of FIG. 33 used in conjunction with the woven filament repair stent 58 of FIG. 32 to increase friction.

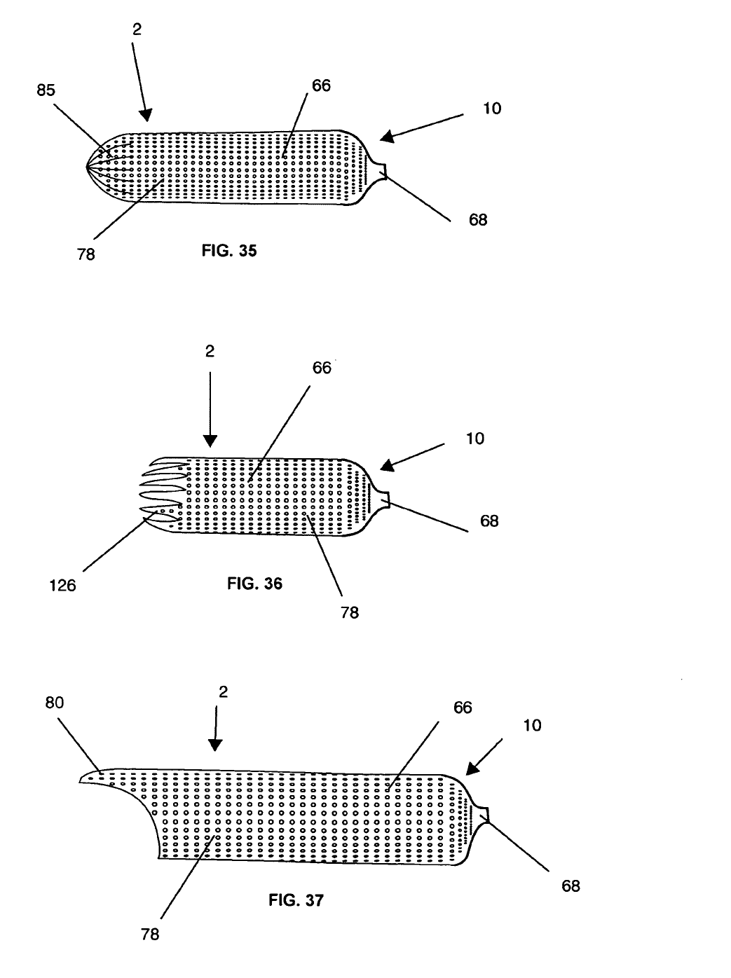

FIGS. 35 through 37 illustrate examples of embodiments of closable tip fracture stents 2 with perforated external walls to increase friction and/or allow fill material 74 injected into the hollow cavity within the stent to leak out, for example for a sealing or cementing purpose or to allow administration of a medicinal preparation to the treatment site, such as a bone growth factor or an antibiotic treatment.

As illustrated by FIGS. 35 through 37 the closable tip fracture stent 2 can also have a deployment tool hole/fill port 68 provided on its trailing end 10 to allow the connection of a deployment tool 48 to the end or a fill tool 72 to the fracture stent. As illustrated by FIG. 38 the deployment tool hole/fill port 68 can be provided with threads 70 or other positive engagement elements such as are generally known in the art. As illustrated by FIG. 38 the deployment tool hole/fill port 68 can accept a deployment tool/fill tool 72. As illustrated by the arrows in FIG. 38 the deployment tool/fill tool 72 can be used to inject a fill material 74 through the tool and through the deployment tool hole/fill port 68 and into the hollow interior cavity of the closable tip fracture stent 2. As further illustrated by FIG. 38, a sealable element, such as the flapper valve 76 illustrated in FIG. 38, can be used to allow the entry of fill material 74 but to prevent its subsequent escape after the deployment tool/fill tool 72 has been removed.

FIGS. 32 and 35 through 37 illustrate examples of embodiments of the closable tip fracture stent 2 with porous outer walls 66. FIG. 32 illustrates that the porous outer wall 66 can comprise a woven interlocking filament. FIGS. 35 through 37 illustrate that the porous outer wall 66 can comprise a wall material having an array of macroscopic 78 or microscopic holes disposed therethrough. (Holes denoted anywhere herein this application as macroscopic holes can also be microscopic holes.) The closable tip fracture stent 2 can also have an outer wall which is made porous by means of microscopic holes.

The closable-tip fracture stent 2 can comprise an expandable linked filament tube enclosed by a wire expandable, plastically deformable cylindrical structure stent for added support. The closable tip fracture stent 2 can also comprise a thin metal screen or wire mesh screen outer shell which can be either integrated into the outer wall of the stent or comprise a separate engageable element to be used in conjunction with the closable tip fracture stent. 2 FIG. 33 illustrates an embodiment of a wire mesh screen that can be slipped over a closable tent fracture stent 2 to increase friction. FIG. 34 illustrates an example of an embodiment of a closable tip fracture stent 2 in conjunction with a wire mesh screen outer sleeve. The wire mesh or thin metal screen can expand and/or open when the closable-tip fracture stent 2 expands.

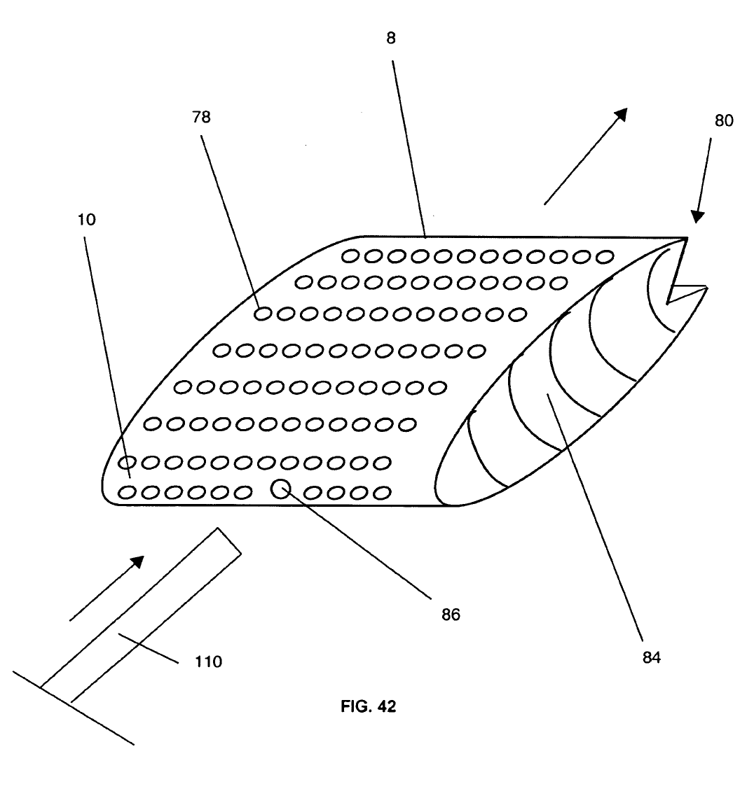

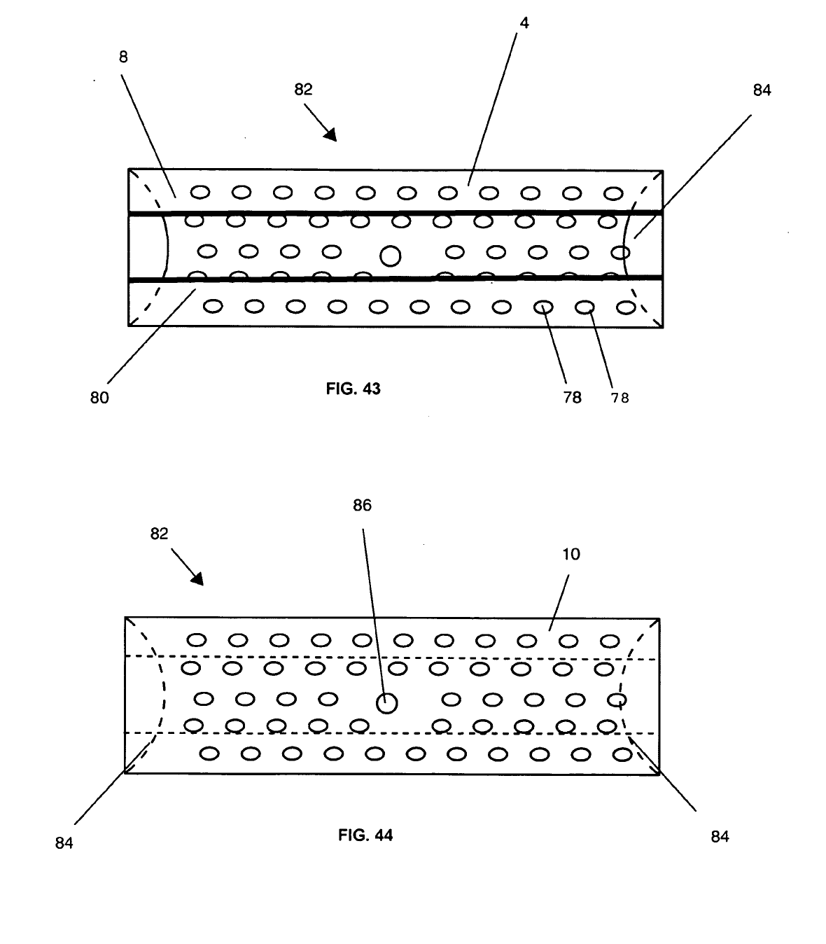

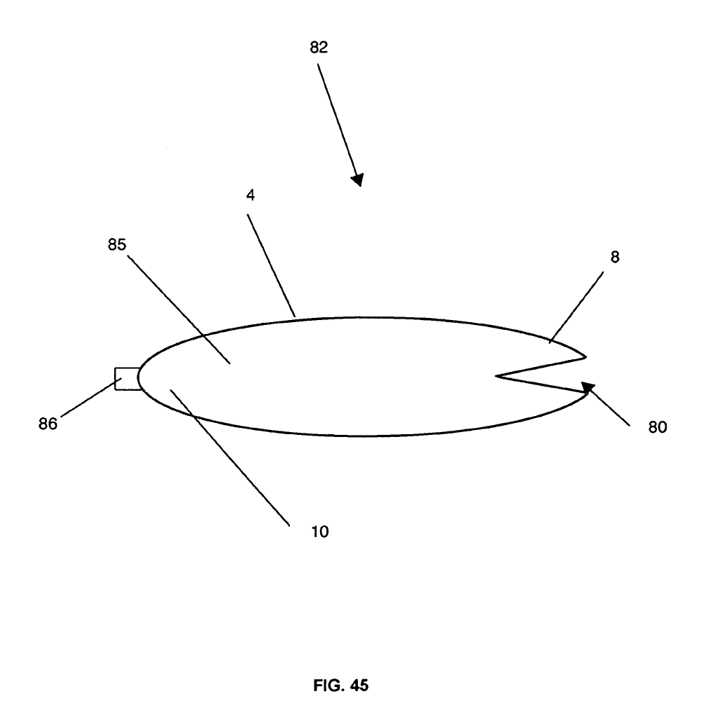

FIGS. 42 through 45 illustrates that the closable tip fractures that can also comprise a flat design. The flat design closable tent fracture stent 82 can have a wall in the shape of a flattened out cylinder. The ends of the cylinder can be closed. As illustrated by FIGS. 42 and 43 the closed end 85 can be flexible to allow the stent to deform in order to conform to the contours of the damage repair site 57. As illustrated by FIGS. 42 through 44 the flexible ends can be concave 84. The closed ends 85 can also be convex or flat. As illustrated by FIGS. 42 through 45 the flat design fracture stent 82 can have a leading 8 and trailing 10 end. The leading end 8 of the flat design closable tip fracture stent 82 can be designed to be open prior to deployment and deform upon itself to close the stent upon deployment. As illustrated by FIG. 42, the exterior wall of the flat design closable tip fracture stent 82 can be porous, for example, as illustrated by FIG. 42, by means of macroscopic holes 78 disposed therethrough. As illustrated by FIGS. 42, 44 and 45 the flat design closable tip fracture stent 82 can also have an insertion tool engagement hole/fill port 86 into which an insertion tool and/or a filling tool 72 can be engaged.

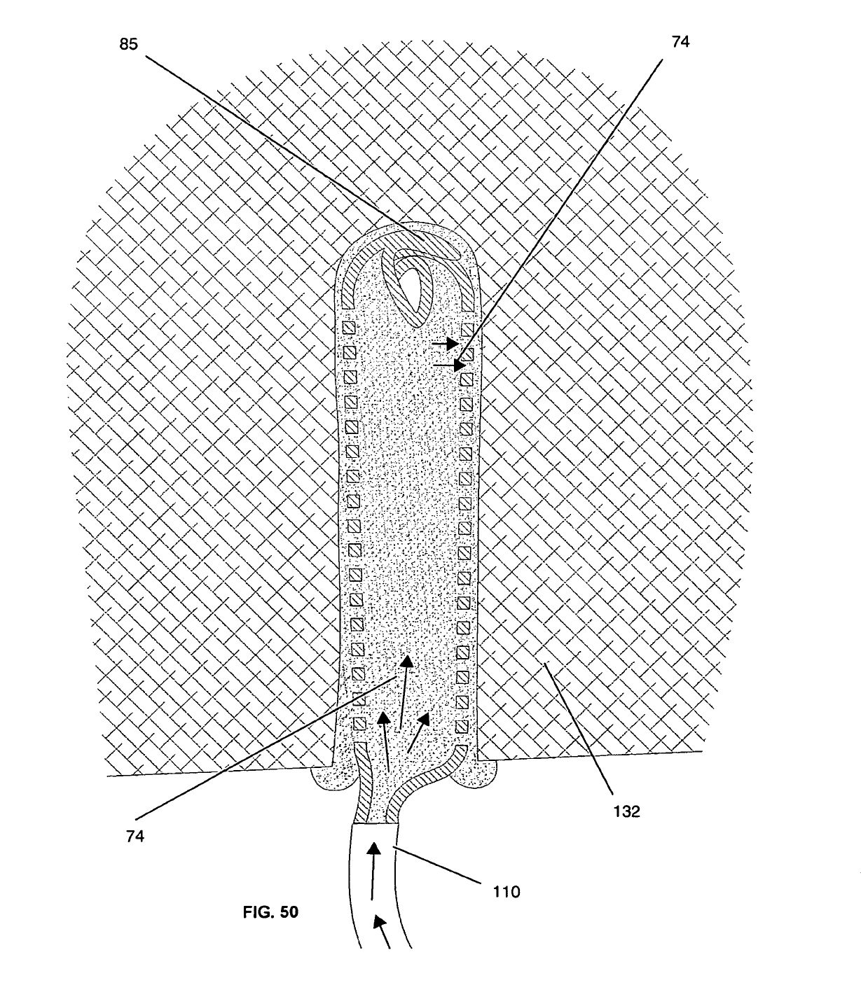

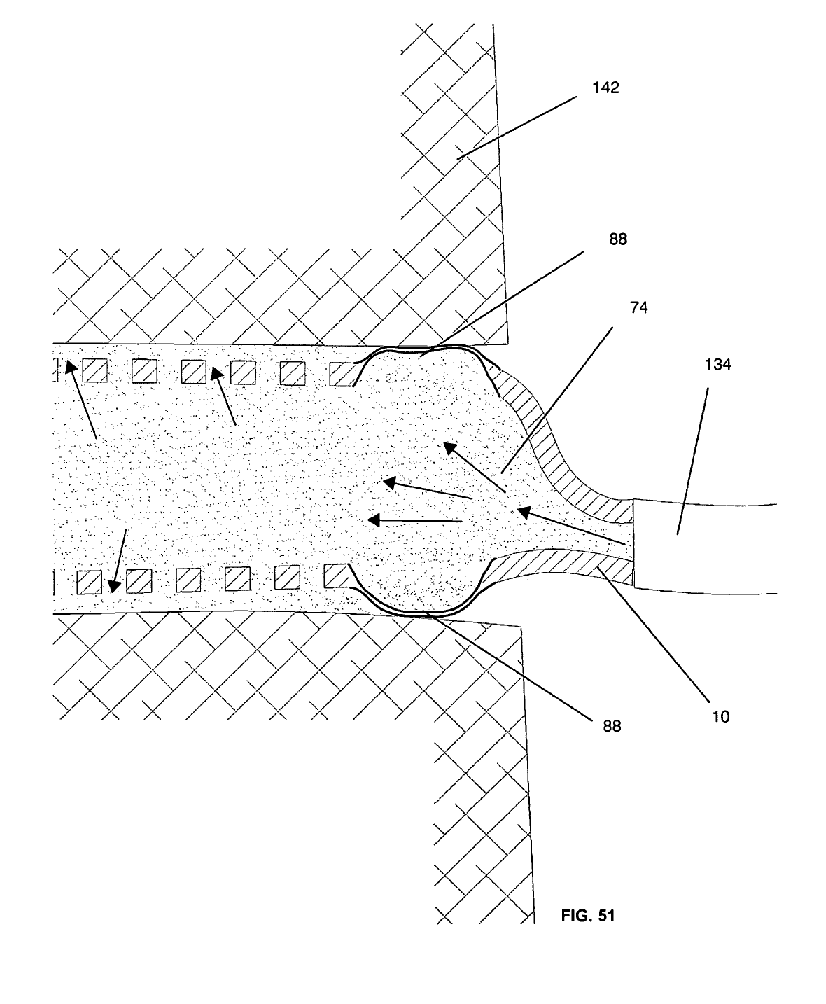

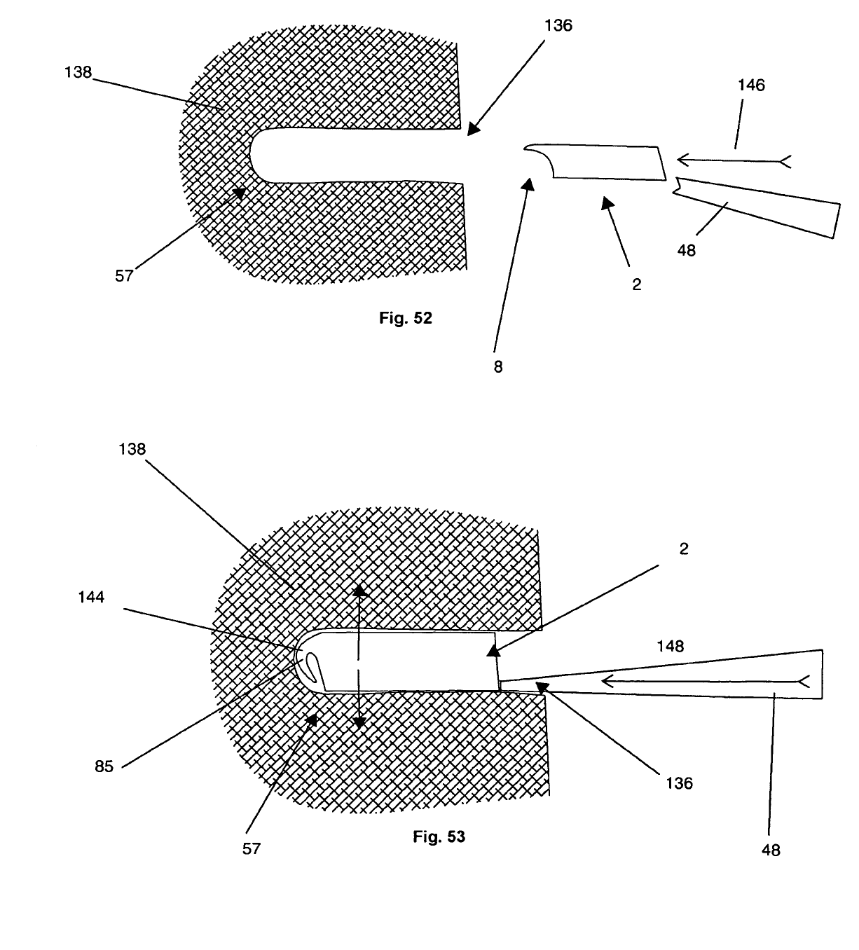

The wall 4 of the stent can have a uniform thickness, or van, in thickness. As illustrated by FIG. 52, the stent can have a thicker wall thickness in areas where less flexibility or expansion is desired, and a thinner wall 88 thickness in areas where greater deformability, or expansion is desired. As illustrated in FIG. 52, the stent can have a thinner wall 88 thickness toward the trailing end 10 in order to exhibit greater circumferential expansion in this area, thereby acting too seal off the repair site 57.

Any or all elements of the expandable support device and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin, Ill. CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET), polyester (e.g., DACRON® from E. I. Du Pont de Nemours and Company, Wilmington, Del.), polypropylene, aromatic polyesters, such as liquid crystal polymers (e.g., Vectran, from Kuraray Co., Ltd., Tokyo, Japan), ultra high molecular weight polyethylene (i.e., extended chain, high-modulus or high-performance polyethylene) fiber and/or yarn (e.g., SPECTRA® Fiber and SPECTRA® Guard, from Honeywell International, Inc., Morris Township, N.J., or DYNEEMA® from Royal DSM N.V., Heerlen, the Netherlands), polytetrafluoroethylene (PTFE), expanded PTFE (ePTFE), polyether ketone (PEK), polyether ether ketone (PEEK), poly ether ketone ketone (PEKK) (also poly aryl ether ketone ketone), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, osteogenic powder, beads of bone) any of the other materials listed herein or combinations thereof. Examples of radiopaque materials are barium sulfate, zinc oxide, titanium, stainless steel, nickel-titanium alloys, tantalum and gold.

Any or all elements of the expandable support device and/or other devices or apparatuses described herein, can be, have, and/or be completely or partially coated with agents and/or a matrix a matrix for cell ingrowth or used with a fabric, for example a covering or sleeve that acts as a matrix for cell ingrowth. The matrix and/or fabric can be, for example, polyester (e.g., DACRON® from E. I. Du Pont de Nemours and Company, Wilmington, Del.), polypropylene, PTFE, ePTFE, nylon, extruded collagen, silicone or combinations thereof.

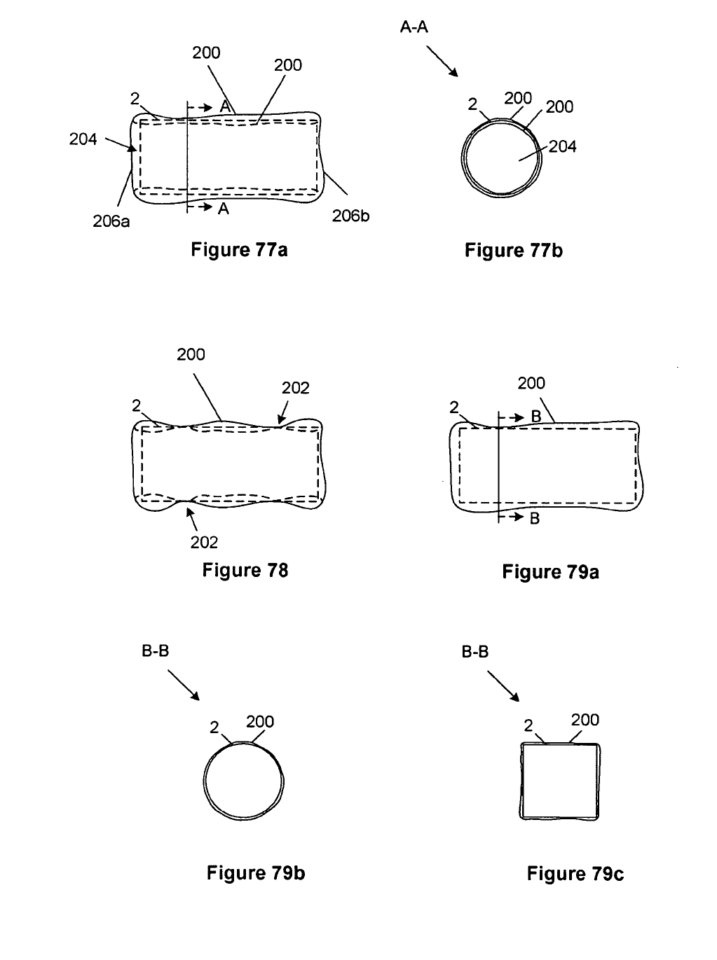

FIGS. 77a and 77b illustrate that the stent 2 can be substantially surrounded by the covering 200. The covering 200 can have a tube configuration. The covering 200 can be separated from the stent 2. For example, the covering 200 can be bound to the stent 2 merely by covering the stent 2 with the covering 200 without directly attaching the stent 2 to the covering 200. Alternatively, as shown in FIG. 78, the stent 2 can be attached to the covering 200 at one or more attachment points 202.

The covering 200 can be wholly integrated with the stent 2. For example, the covering 200 can be fused or fixedly attached to the stent at substantially all points on the stent-facing surface of the covering 200.

As shown in FIGS. 77a and 77b, the covering 200 can have a center channel 204 that can be accessible by front and/or rear covering ports 206a and 206b. The covering 200 can have the front and/or rear covering port 206a and/or 206b. The covering 200 can wrap around the edges of the stent 2 in close proximity of all sides of the walls of the stent 2. As shown in FIGS. 79a and 79b, the covering 200 can have no covering ports (i.e., the covering 200 may not wrap around the insides of the walls of the stent 2). The center channel can be substantially inaccessible without rupturing, osmotic delivery though, or injecting through the covering 200.

FIGS. 77b and 79b illustrate that the stent 2 and/or covering 200 can have substantially circular or oval transverse cross section. FIG. 79c illustrates that the stent 2 and/or covering 200 can have substantially square or rectangular cross-sections.

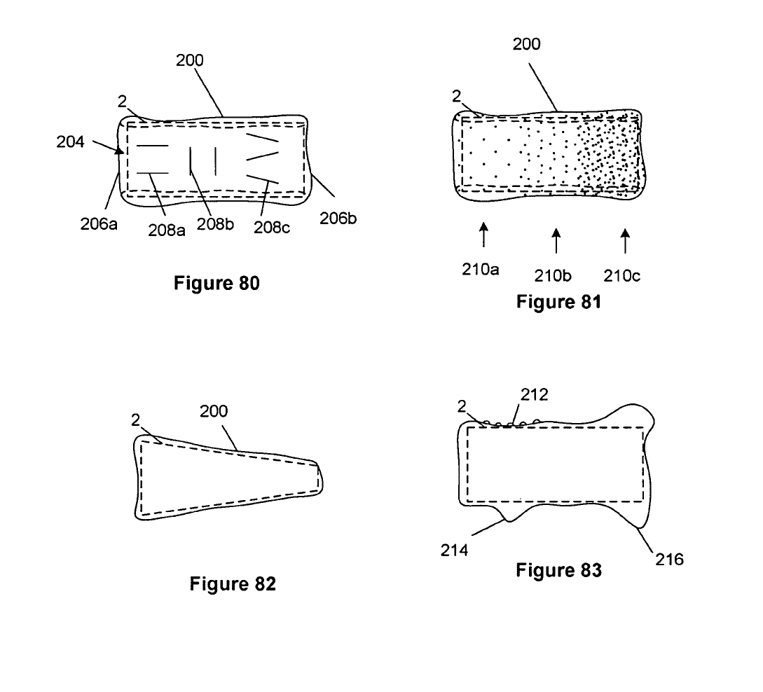

FIG. 80 illustrates that the covering 200 can have slits 208. The slits 200 can be, for example, oriented as longitudinal slits 208a, latitudinal or transverse slits 208b, angled slits 208c, or combinations thereof. The multiple slits 208 can be configured in transverse rows and/or longitudinal columns along the covering 200. The slits 208 can be in a closed configuration when the stent 2 is in a radially contracted configuration. The slits 208 can be on the radially-outward facing wall of the covering 200, as shown, and/or on one or more of the longitudinally-outward facing end walls. The slits 208 can be in an opened configuration when the stent is in a radially expanded configuration. The coverings 200 can have struts and/or fibers. The covering can be a screen, for example a metal screen, a mesh screen, a wire screen, or combinations thereof. The coverings 200 can have or be films, for example slitted films.

The covering 200 can be made from any of the materials described herein, including plastics, metals, ceramics, other materials, and combinations thereof.

The covering 200 can be fabric. The covering 200 can be knitted, woven, braided, or combinations thereof.

The covering 200 can be deformable and/or resilient. For example, the covering 200 can resiliently expand and contract with radial expansion and contraction of the stent 2. The covering 200 can deformably expand with the radial expansion of the stent 2. The covering 200 can be mechanically expandable (e.g., due to the force exerted by the stent 2 during radial expansion of the stent 2). The covering 200 can be self-expandable. For example, resilient fibers or wires can be woven, knitted or braided into the covering 200.

The covering 200 can be filled before or after delivery to a target site and/or radial expansion. The covering 200 can be coated or filled with any material disclosed herein or combinations thereof, for example agents or fillers disclosed, infra.

The covering 200 can be porous or non-porous. The pores can be microscopic holes and/or macroscopic holes. The covering 200 can have porosity that varies based on location on the covering 200. For example, the covering 200 can have porosity that can vary with respect to longitudinal position on the covering 200. FIG. 81 illustrates that the covering 200 can have a first porosity zone 210a having a first porosity, a second porosity zone 210b having a second porosity, and a third porosity zone 210c having a third porosity. The first porosity zone 210a can be at one end of the covering 200. The second porosity zone 210b can span the longitudinal center of the covering 200. The third porosity zone 210c can be at the second end of the covering 200. As shown in FIG. 81, the first porosity can be less than the second porosity, and the second porosity can be less than the third porosity. The second porosity and third porosity can be greater than, less than or equal to the first porosity.

The porous covering and can be configured to permit a fill material injected into the hollow cavity inside of the covering 200 and/or stent 2 to leak out of the hollow cavity.

The covering 200 can have a porosity that can contain fluids until a first pressure inside and/or outside of the covering is reached. When the covering 200 is exposed to the first pressure, the porous covering 200 can allow fluid flow through the covering 200.

FIG. 82 illustrates that the stent 2 and/or covering 200 can be shaped with a radial taper with respect to the longitudinal axis of the stent 2 and/or covering 200.

FIG. 83 illustrates that the covering 200 can have a texture, such as bumps 212 or loops over part (e.g., the radially outer-facing surface) or all of the surface of the covering 200. The covering 200 can have a deformable or rigid bulge 214 that can extend radially. The covering 200 can have a dogbone configuration 216, for example, radial bulging in two or more directions at one or both ends of the covering 200. The covering 200 can have a configured to match the configuration of the underlying stent 2 or have a configuration that does not substantially match all or part of the stent 2.

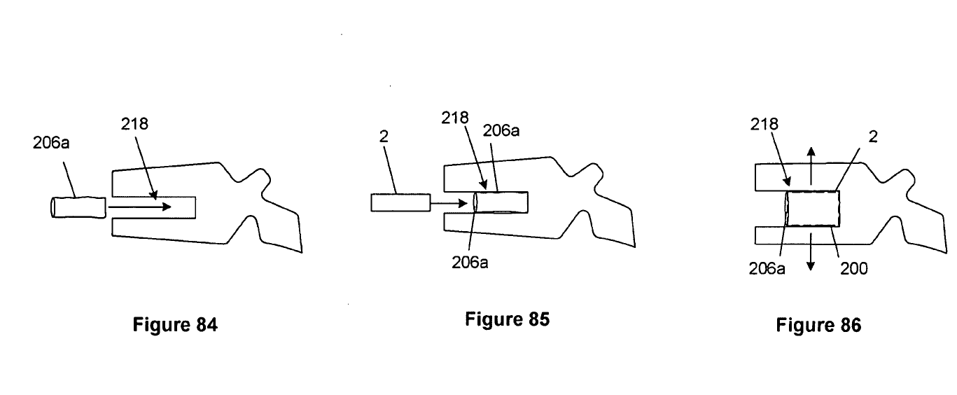

During use the covering 200 can be deployed to the target site attached to the stent 2. During use, the covering 200 can be deployed to the target site separately from the stent 2. For example, as shout in FIG. 84, the covering 200 can be inserted into a prepared (e.g., reamed or drilled, if necessary) target site 218. The target site 218 can be in a fracture in a bone, for example in a compression fracture in a vertebra. FIG. 85 illustrates that the stent 2 can be inserted into the target site 218 and through the front covering port 206a into the covering 200. FIG. 86 illustrates that the stent 2 and covering 200 can then be actively or passively radially expanded. For example, the stent 2 can self-expand or be deformably expanded by a deployment tool, resulting in the covering 200 being passively expanded outside of the stent 2. The target site 218 can radially expand, for example substantially restoring the target site 218 to the natural or a more beneficial anatomical configuration of the target site 218.

A filler can be inserted into the radially expanded stent 2 and/or covering 200, for example through the front port 206a, and/or injected through the covering 200. The filler can then elute or otherwise disperse out of the stent 2 and/or covering 200, for example through pores in the covering 200.

The expandable support device and/or elements of the expandable support device and/or other devices or apparatuses described herein and/or the fabric can be filled, coated, layered and/or otherwise made with and/or from cements, fillers, glues, and/or an agent delivery matrix known to one having ordinary skill in the art and/or a therapeutic and/or diagnostic agent. Any of these cements and/or fillers and/or glues can be osteogenic and osteoinductive growth factors.

Examples of such cements and/or fillers includes bone chips, demineralized bone matrix (DBM), calcium sulfate, coralline hydroxyapatite, biocoral, tricalcium phosphate, calcium phosphate, polymethyl methaciylate (PMMA), biodegradable ceramics, bioactive glasses, hyaluronic acid, lactoferrin, bone morphogenic proteins (BMPs) such as recombinant human bone morphogenetic proteins (rhBMPs), other materials described herein, or combinations thereof.

The agents within these matrices can include any agent disclosed herein or combinations thereof, including radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc. Whitehouse Station, N.J.; CELEBREX® from Pharmacia Corp., Peapack, N.J. COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, Sp1 Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

The closable-tip fracture stents 2 can be laser cut, or non-laser cut. The closable-tip fracture stent 2 can be molded, cast, sintered, or extruded. The closable-tip fracture stent 2 can be laser cut in a partially opened pattern, then the closable-tip fracture stent 2 can be loaded (e.g., crimped) onto a deployment tool 48.

The closable-tip fracture stent 2 can be longitudinally segmented. Multiple closable-tip fracture stents can be attached leading end 8 to trailing end 10, and/or a single closable-tip fracture stent can be severed longitudinally into multiple closable-tip fracture stents.

### Method of Use

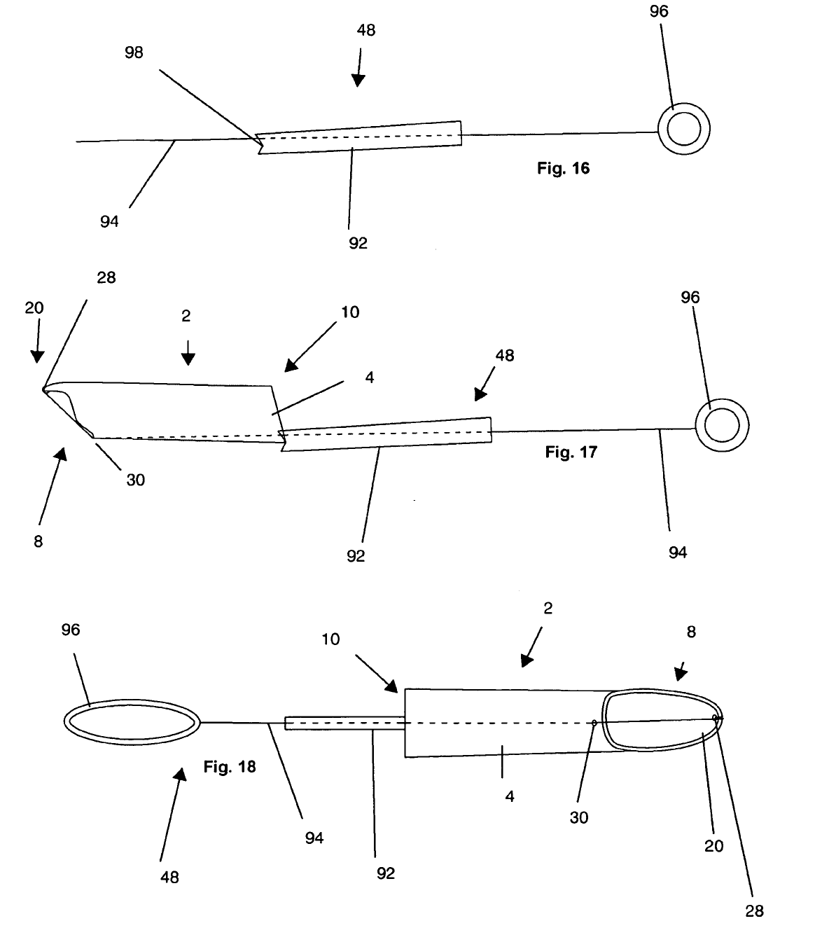

FIG. 16 illustrates a deployment tool 48 onto which the closable-tip fracture stent 2 can be loaded in a open (i.e., uncontracted) configuration. The deployment tool 48 can have a handle 92 with a cable 94 fixed at its end to a grip 128, for example a lever or a pull ring 96. The deployment tool 48 can have an engagement notch 98 to engage and grip the trailing end 10 of the closable-tip fracture stent 2, for example in order to manipulate the closable-tip fracture stent 2 during deployment. As illustrated in FIGS. 17 and 18, the cable 94 can lead from a pull ring 96 slidably through a handle 92 to a fixation point at the tip 20 of the closable-tip fracture stent 2. The cable 94 can slidably pass through an intermediate eyelet in the closable-tip fracture stent 2, for example in the wall of the closable-tip fracture stent at a point opposite the tip, for example a second draw eyelet 30 as illustrated in FIGS. 17 and 18. The distal end of the cable 94 can be removably attached to a draw eyelet on the tip 20 of the closable-tip fracture stent 2, for example a first draw eyelet 28 as illustrated in FIGS. 17 and 18.

The cable 94 can also attach to one or more of the distal ends of the crown points 32 on a closable-tip fracture stent 2 with a crowned leading end.

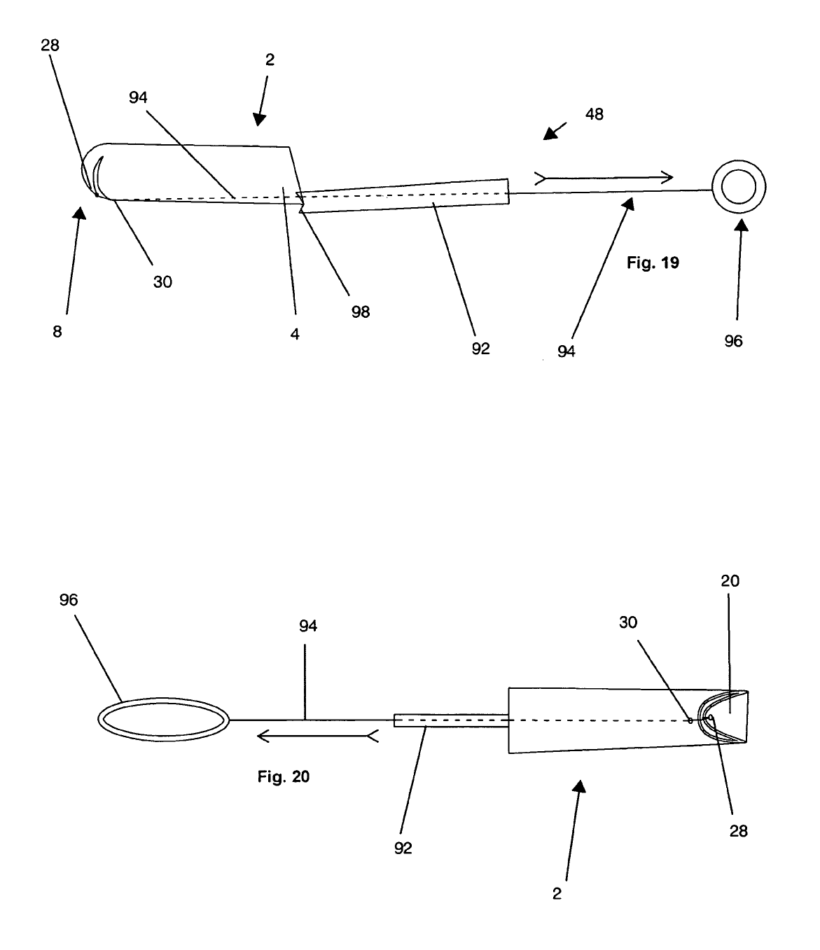

FIGS. 19 and 20 illustrate that a pull ring 96 of the deployment tool 48 can be pulled to withdraw the cable 94 through a handle 92 and through the second draw eyelet 30, thereby causing the tip of the closable-tip fracture stent 2 to deform and close upon itself, sealing the leading end 8 of the closable-tip fracture stent 2. This action can also expand the closable-tip fracture stent 2 in height, diameter, or profile. Use of a closable tip fracture stent 2 with a draw eyelet/cable closure system can be useful, for example, in situations where it is not desirable to deploy the fracture stent completely against the end of the repair site 57. In such cases, the closable tip 80 of the fracture stent can be closed by use of the cable insertion/deployment tool.

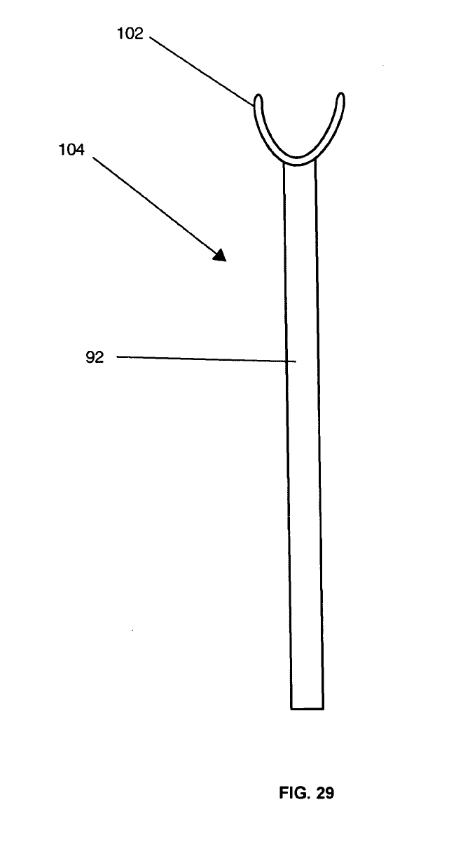

FIG. 29 illustrates an embodiment of a push-type deployment tool that can be used to insert and deploy closable tip fracture stents 2 having closable tips 80 on both leading and trailing ends. As illustrated by FIG. 29 a curved tip insertion/deployment tool 104 for use with closable tip fracture stents 2 having closable tips both on the leading 106 and trailing 108 ends can have a curved or parabolic distal end 102. As illustrated by FIG. 29, the curved tip tool 104 can also have a handle 92. The curved or parabolic distal end 102 can be shaped to close the trailing end of the closable tip fracture stent 2 upon deployment when the curved or parabolic tip 102 is forced against the trailing end 10 of the closable tip fracture stent 2. This action can cause the closable tip 80 on the trailing end 10 to plastically deform and seal off the end of the closable tip fracture stent 2 while simultaneously forcing the closable tip fracture stent 2 into the repair site 57, thereby causing the closable tip 80 on the leading end 8 of the stent to also close upon itself. This type of curved tip push tool 104 can also be used to deploy a closable-tip fracture stent 2 with a ratchet closing mechanism as illustrated in FIG. 26.

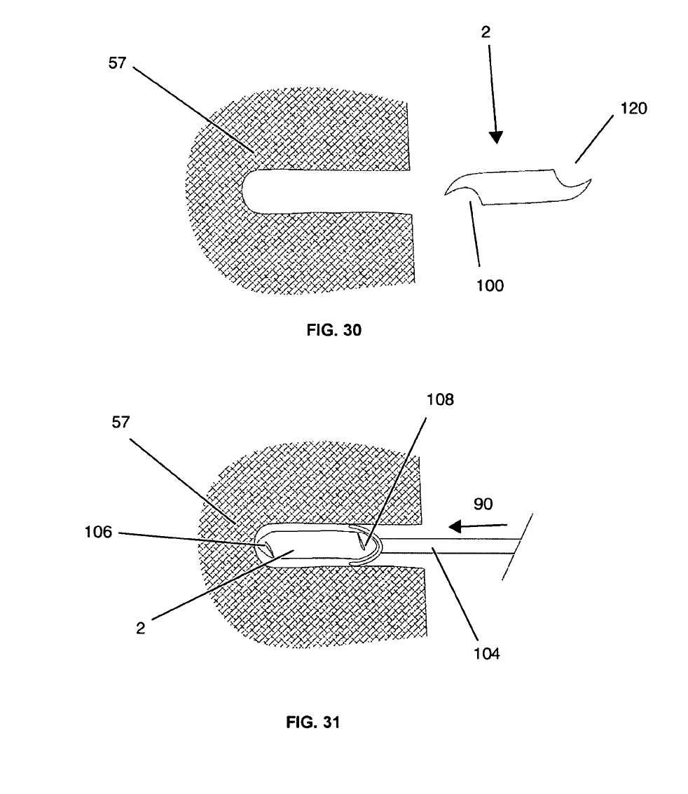

FIGS. 30 and 31 illustrate how a curved tip tool 104 can be used to deploy a closable tip fracture stent 2 having closable tips 80 on both ends. FIG. 30 illustrates how the closable tips on the leading and trailing ends are both open (100, 120) while the stent is undeployed. FIG. 31 illustrates how the closable tip on the leading end deforms to close upon itself 106 in response to the force applied by the curved tip insertion/deployment tool 104, illustrated by the arrow 90 in FIG. 31. FIG. 31 also illustrates how the closable tip on the trailing end of the stent closes upon itself 108 due to the action of the curved or parabolic tip being forced against the trailing end of the stent.

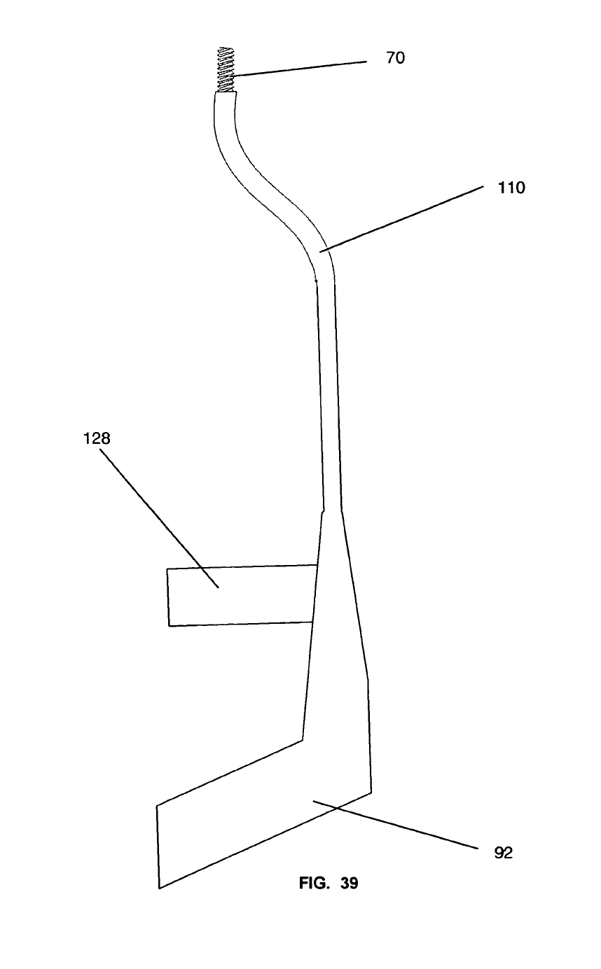

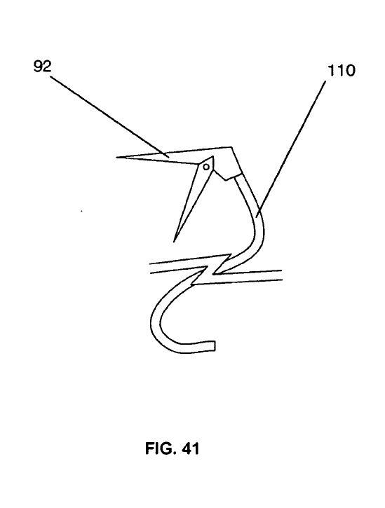

FIGS. 39, 40 and 41 illustrate three examples of embodiments of deployment tools that can be used to deploy the closable tip fracture stent 2 into a repair site 57 in a damaged bone. As illustrated by FIGS. 39, 40, and 41, the deployment tool 48 can have a elongated deployment extension 110. The elongated deployment extension 110 can be flexible and/or steerable by the operator. The elongated deployment extension 110 can be extendable or fixed in length. The elongated deployment extension 110 can have a camera or other orthroscopic device of fixed thereto.

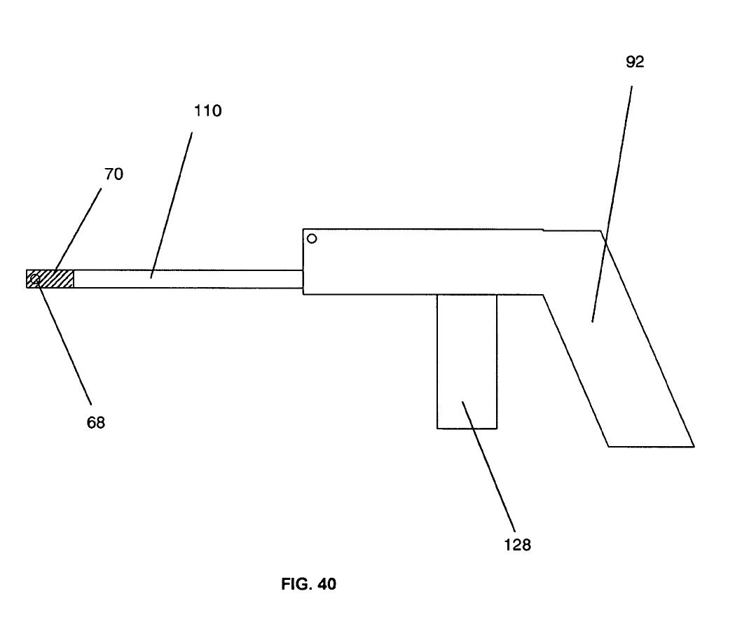

As illustrated by FIGS. 39 and 40 the distal end of the elongated deployment extension 110 can have a engageable element for engaging the closable tip fracture stent 2. The engageable element can comprise a threaded element 70 or another secure attachment means as is commonly known in the art. As illustrated by FIG. 38 the elongated deployment extension 110 can have a conduit 112 or passageway therethrough, for example to allow the injection of a fill material 74 from the tool into the engaged stent.

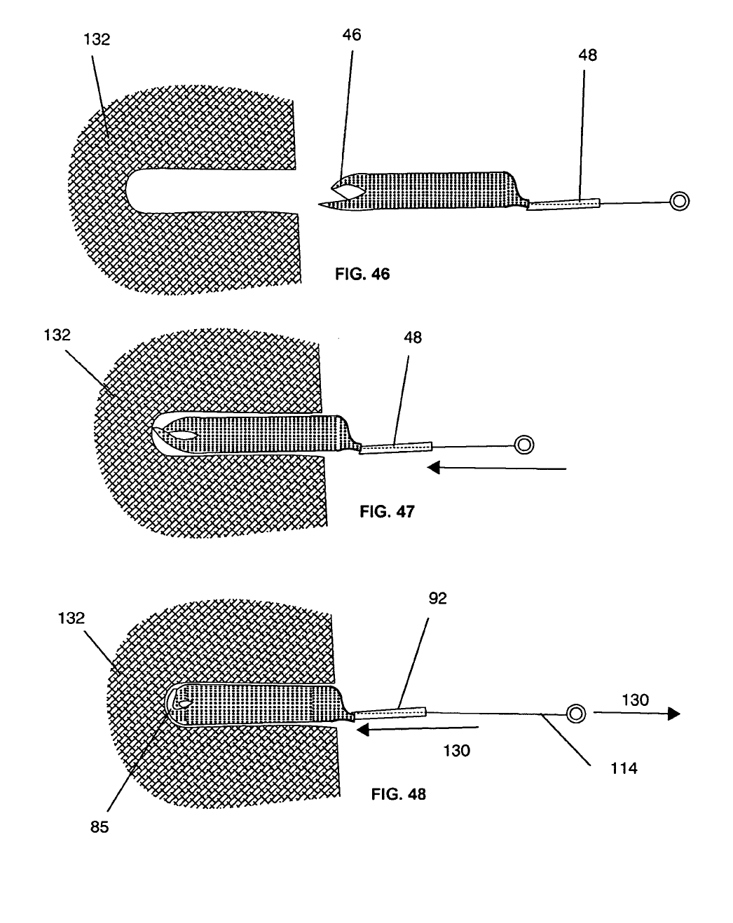

FIGS. 46, 47 and 48 illustrate the deployment of an embodiment of the closable tip fracture stent 2. As is illustrated by FIG. 46, the fracture stent is connected to the deployment tool 48 prior to deployment into the repair site in the bone 132. As FIG. 46 illustrates, prior to deployment, the closable tip of the fracture stent is open 126. FIG. 47 illustrates how the fracture stent can be inserted into the damage site in the bone 132 using the deployment tool 48. The black arrow 130 in FIGS. 47 and 48 indicate the direction of motion and force. FIG. 47 illustrates how the closable tip of the fracture stent starts to fold onto itself and close when the leading end of the stent comes into contact with the terminal end of the prepared repair site in the bone 132. FIG. 48 illustrates how the closable tip of the fracture stent 2 closes completely, sealing the end of the fracture stent.

FIG. 48 also illustrates how a deployment tool of the type illustrated in FIGS. 16 through 20 can be used to fully close the closable tip 80 of the fracture stent. As indicated by the black arrows 130 in FIG. 48 the deployment cable 114 of the insertion tool 134 which is connected via its distal end to the closable tip 80 of the fracture stent can be withdrawn with a force opposite in direction to the force used on the handle 92 of the deployment tool 48 to insert the fracture stent. This withdrawal of the deployment cable 114 can further cause the closable tip 80 of the fracture stent to completely close. This may be desirable, for example, in cases where the fracture stent is not to be deployed completely against the terminus of the repair site in the bone 132. In such cases, the closable tip 80 of the fracture stent can be closed by use of the insertion tool 134.

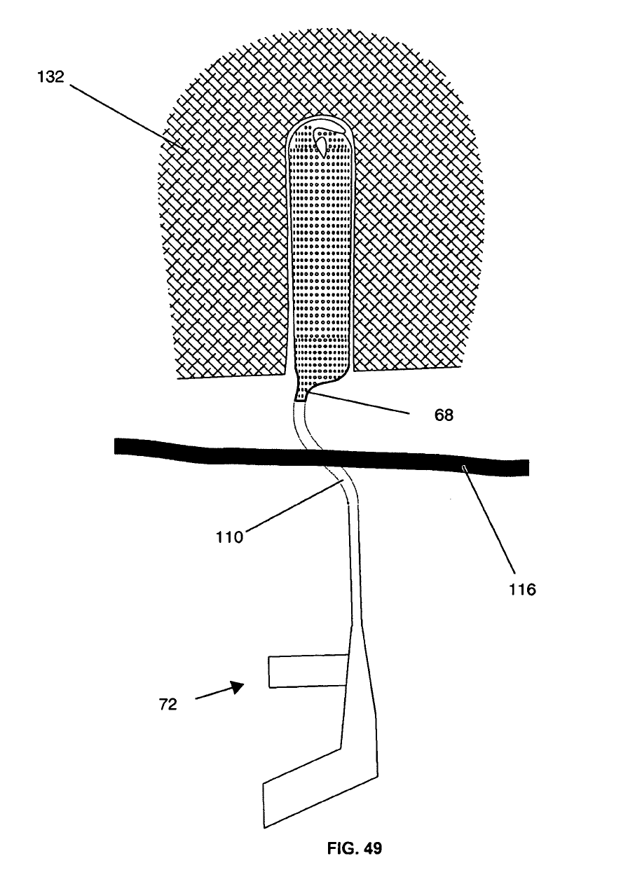

FIG. 49 illustrates how the elongated deployment extension 110 of the deployment tool/fill tool 72 can be inserted through the skin 116 of the patient to engage the closable tip fracture stent 2 by means of the deployment tool hole/fill port 68.

FIG. 50 illustrates how a fill material 74 can be injected into the closable tip fracture stent 2 through the deployment tool/fill tool 72. As the black arrows in FIG. 50 illustrate, the fill material 74 can pass through the conduit passageway within the elongated deployment extension 110 of the deployment tool 48 and completely fill the fracture stent. As is further illustrated by FIG. 50 the fill material 74 can pass through the porous walls 66 of the fracture stent to come into direct contact with the inner surface of the repair site 57, for example to secure the fracture stent in place and/or promote healing or inhibit infection.

As is illustrated by FIG. 51, the closable tip fracture stent 2 can have a thinner wall 88 toward the proximal end of the stent. This can allow the proximal end of the stent in the area of the thinner wall 88 to expand in response to an injection of fill material 74, to a greater degree than the distal portion of the stent, thereby sealing the stent in the repair site 57 and preventing the escape of the fill material 74 into the body of the patient beyond the repair site 57.

FIGS. 52 and 53 illustrate how the closable tip fracture stent 2 can be deployed into a repair site in bone 132. FIG. 52 illustrates that the closable tip 80 of the fracture stent 2 can be open prior to deployment. FIG. 52 illustrates that the closable-tip fracture stent 2, for example in an open configuration, can be loaded on a deployment tool 48, for example a push-type deployment tool. The trailing end 10 of the closable-tip fracture stent 2 can be received by and/or interference fit in the distal end of the deployment tool 48, for example by connection to an engagement notch 98. After the closable-tip fracture stent 2 has been deployed, the deployment tool 48 can be disengaged from the closable-tip fracture stent 2 and withdrawn from the repair site 57.

FIG. 53 illustrates that the closable tip fracture stent 2 can close in response to the force 148 of being pushed against the terminal end of the repair site 57. FIG. 53 also illustrates how the deformation of the body of the fracture stent resulting from the closable tip 85 folding upon itself can cause the expansion 150 of the diameter or circumference of the fracture stent, thereby securing the stent in place in the repair site 57.

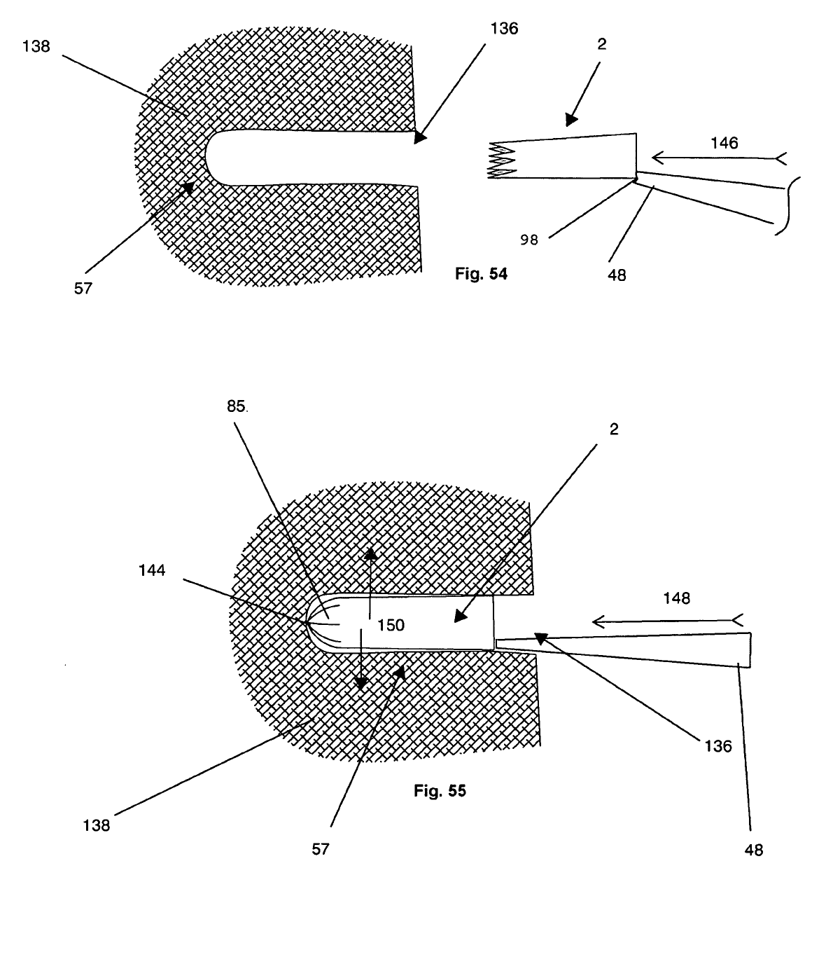

FIGS. 54 and 55 illustrate how a closable tip fracture stent 2 having a crowned tip 36 can be deployed into a repair site in bone 132. FIG. 54 illustrates how the closable tip fracture stent 2 can be open prior to deployment. FIG. 54 further illustrates how the closable tip fracture stent 2 can be connected to the deployment tool 48 by means of an engagement notch 98 and maneuvered into the repair site 57 by use of the deployment tool 48. FIG. 55 illustrates how the closable tip 80 of the fracture stent can close in response to being forced against the terminal end of a prepared access port 136 in the repair site 57. An access port 136 can be created in the repair site of the bone 132, for example, by use of an orthopedic drill.

FIG. 55 illustrates that the deployment of the closable-tip fracture stent 2 can cause its expansion 150, for example in height, diameter, and/or profile, to engage the tissue to be repaired. FIG. 55 further illustrates how the diameter and/or circumference of the fracture stent can increase in response to the deformation of the closable tip 80 of the fracture stent; thereby securing the fracture stent in the repair site 57. As illustrated by FIG. 55, as the crown points 32 deform so as to contact each other, further force on the deployment tool 48 can cause the closable-tip fracture stent 2 to expand to engage the repair site 57.

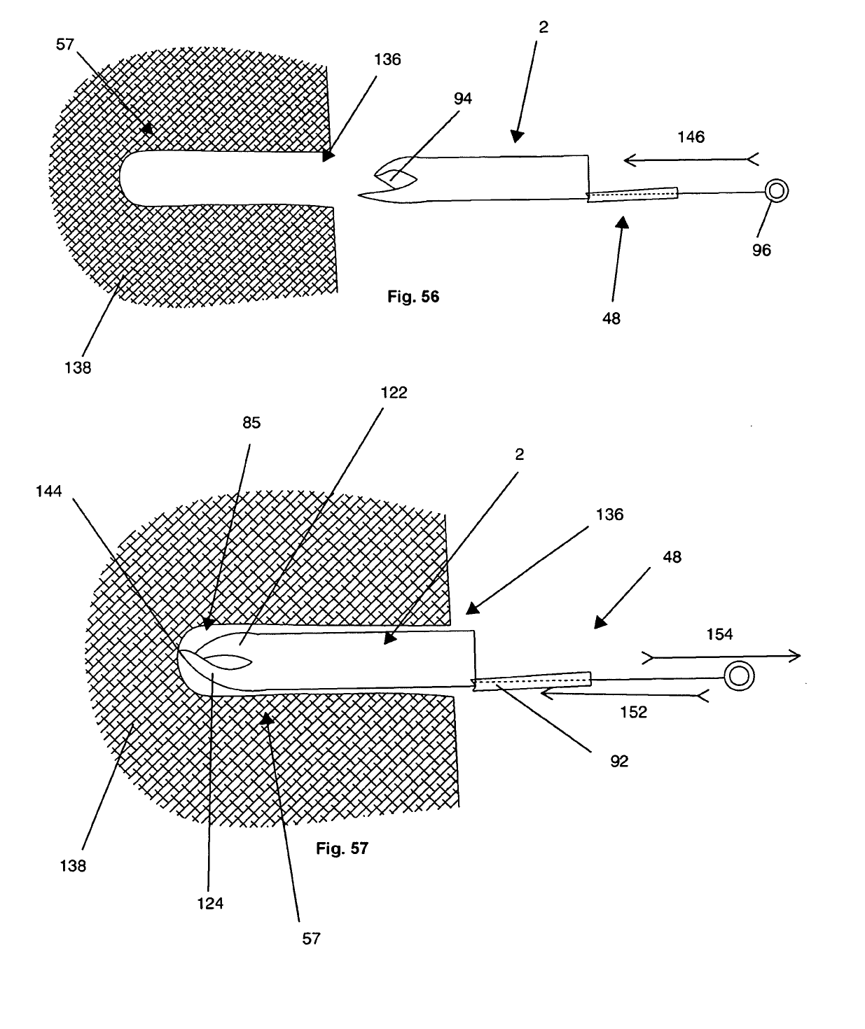

FIGS. 56 through 58 illustrate how a closable tip fracture stent 2 with a crowned tip 36 having to crowns of unequal lengths can be deployed into a repair site in a bone 132. As FIG. 56 illustrates, prior to deployment; the fracture stent can be connected to the deployment tool 48 and maneuvered toward an access port 136 prepared in the bone at the repair site 132. FIG. 57 illustrates how the fracture stent can be inserted, by means of application of force on the handle 152 of the deployment tool 48, into the access port 136 created at the repair site in the bone 132. FIG. 57 further illustrates how the long crown 124 of the fracture stent can begin to fold back upon itself in response to contacting the access port end 144. FIG. 57 further illustrates how the short crown 122 of the repair stent can be folded back inside the long crown 124 by use of the deployment cable 114. FIG. 57 illustrates how pulling on the deployment cable 114 in a direction 154 opposite the direction of insertion of the fracture stent can pull the short crown 122 of the fracture stent back onto itself, thereby closing the fracture stent.

FIG. 58 illustrates how the fracture stent can be completely closed by a combination of being forced against the end of the access port 136 with the deployment tool handle 92 and by closing the short crown 122 by pulling 154 on the deployment cable 114.

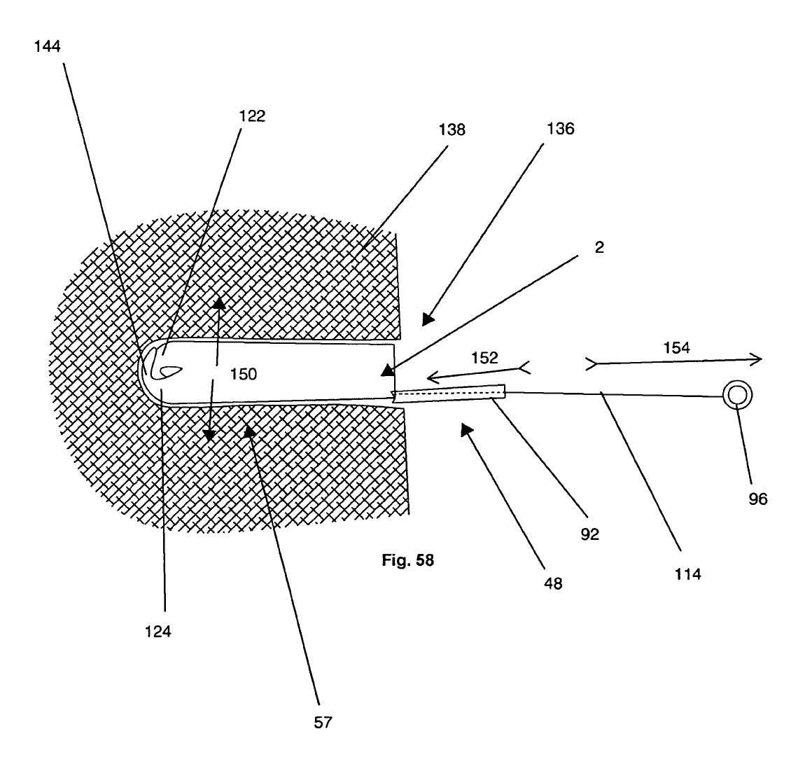

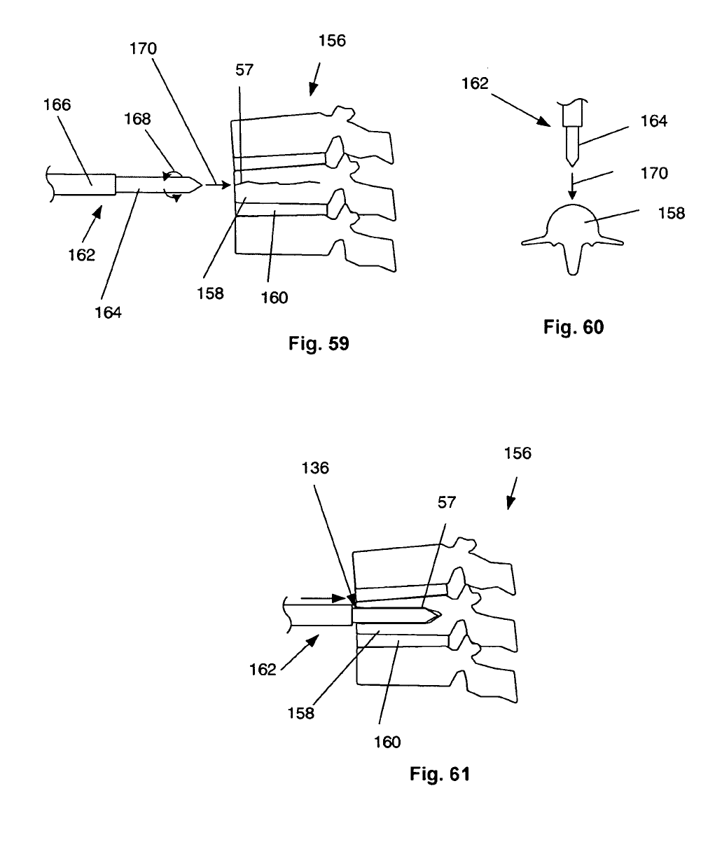

FIGS. 59 (side view) and 60 (top view) illustrate a vertebral column 156 that can have one or more vertebra 158 separated from the other vertebra by discs 160. The vertebra 158 can have a damage site 57, for example a compression fracture. As illustrated in FIGS. 59 through 61, an access tool 162 can be used to gain access to the damage site 57 and or increase the size of the damage site 57 to allow deployment of the closable-tip fracture stent 2 therein. The access tool 162 can be a rotating or vibrating drill 164 that can have a handle 166. The drill 164 can be operating, as shown by arrows 168. The drill can then be translated, as shown by arrow 170, toward and into the vertebra 158 so as to pass into the damage site 57.

FIG. 61 illustrates that the access tool can be translated, as shown by the arrow, to remove tissue at the damage site. The access tool can create an access port 136 at the surface of the vertebra The access port 136 can open to the damage site. The access tool can then be removed from the vertebra.

FIG. 62 illustrates a cracked vertebra 172 in a spinal column 174 prior to the creation of a access port 136 at the damage site 57. FIG. 63 illustrates an access port 136 created by the method described in FIGS. 59 through 61, at the damage site 57.

The vertebra 158 can have multiple damage sites and closable-tip fracture stents 2 deployed therein. The closable-tip fracture stents 2 can be deployed from the anterior, posterior, both lateral, superior, inferior, any angle, or combinations of the directions thereof.

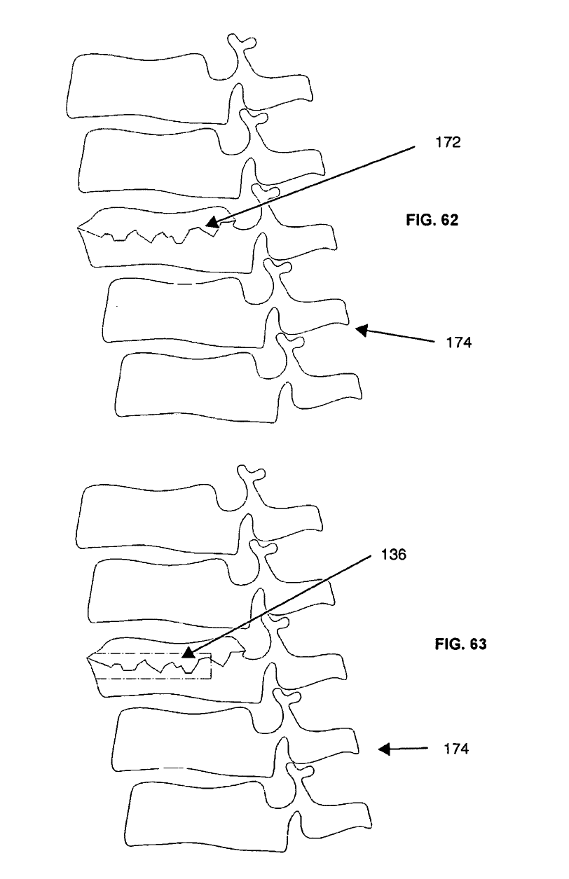

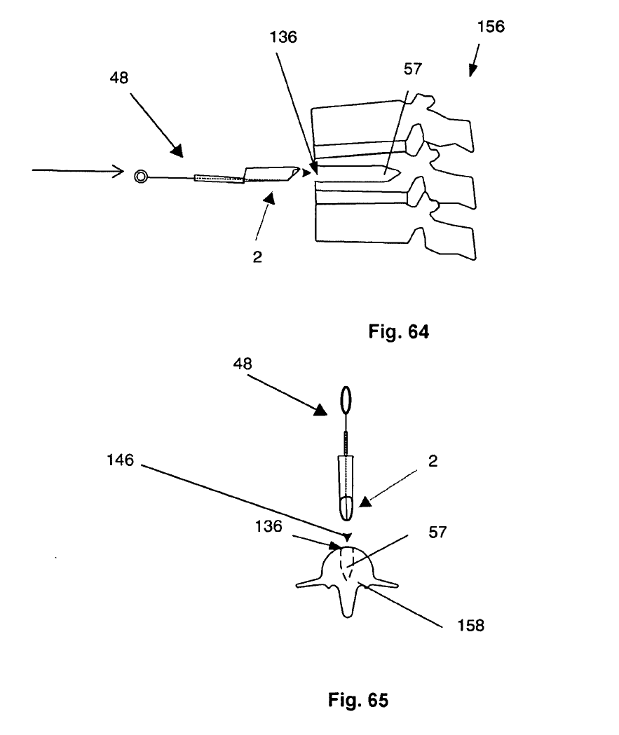

The closable-tip fracture stent 2 can be used to repair damage sites, for example in the vertebral column 156. FIGS. 64 and 65 illustrate translating, as shown by arrows 146, the deployment tool 48 loaded with the closable-tip fracture stent 2 through the access port 136 from the anterior side of a vertebral column 156.

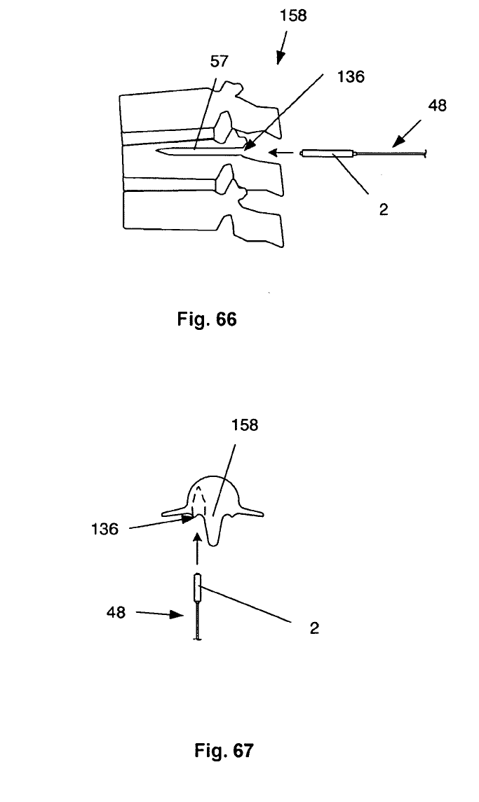

FIGS. 66 and 67 illustrate translating, as shown by arrows 146, the deployment tool 48 loaded with the closable-tip fracture stent 2 through the access port 136 from the posterior side of a vertebral column 156.

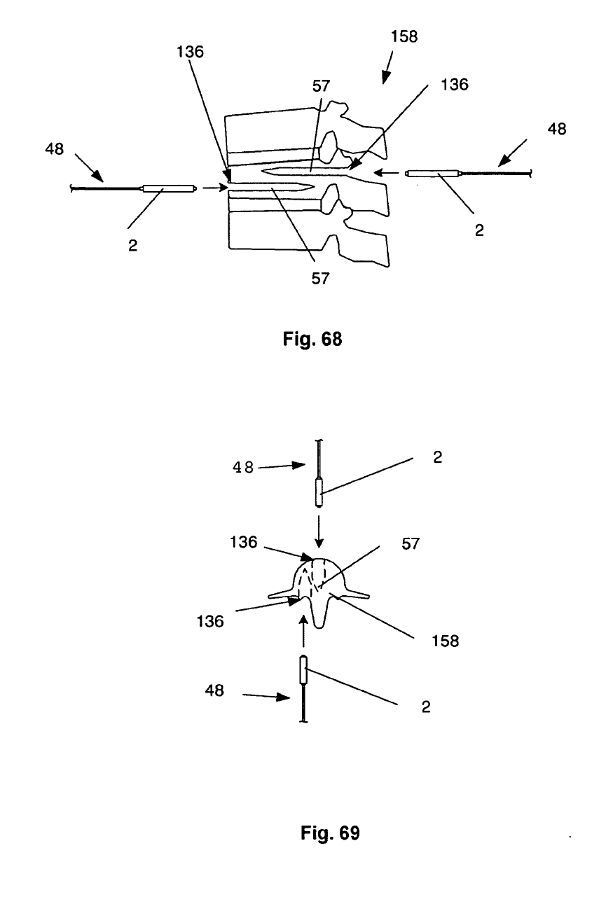

More than one fracture stent can be deployed to a damage site 57. In cases where more than one fracture stent is deployed, different fracture stents can be deployed in different manners. FIGS. 68 and 69 illustrate translating, as shown by arrows, more than one deployment tool 48 loaded with the more than one closable-tip fracture stents through access ports 136 from the posterior side and anterior side of a vertebral column 156.

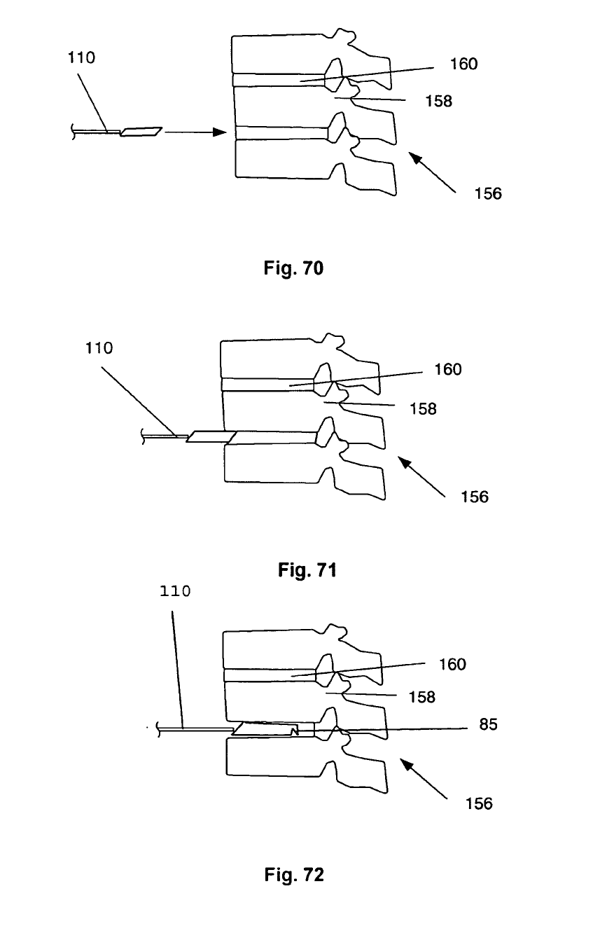

FIGS. 70, 71 and 72 illustrate closable-tip fracture stents 2 can be used to repair soft tissue, for example a herniated disk in a spinal column 156. FIG. 78 illustrates translating, as indicated by the arrow, a deployment tool 48 loaded with a closable tip fracture stent 2, toward a herniated disk. FIG. 71 illustrates that a deployment tool 48, for example a push type deployment tool, can be used to insert a closable tip fracture stent 2 into a damage site 57, for example a herniated disk in a vertebral column 156. FIG. 72 illustrates that the closable tip 80 on the leading end of the fracture stent can close in response to being forced into the repair site 57 with a deployment tool 48, for example a push type deployment tool.

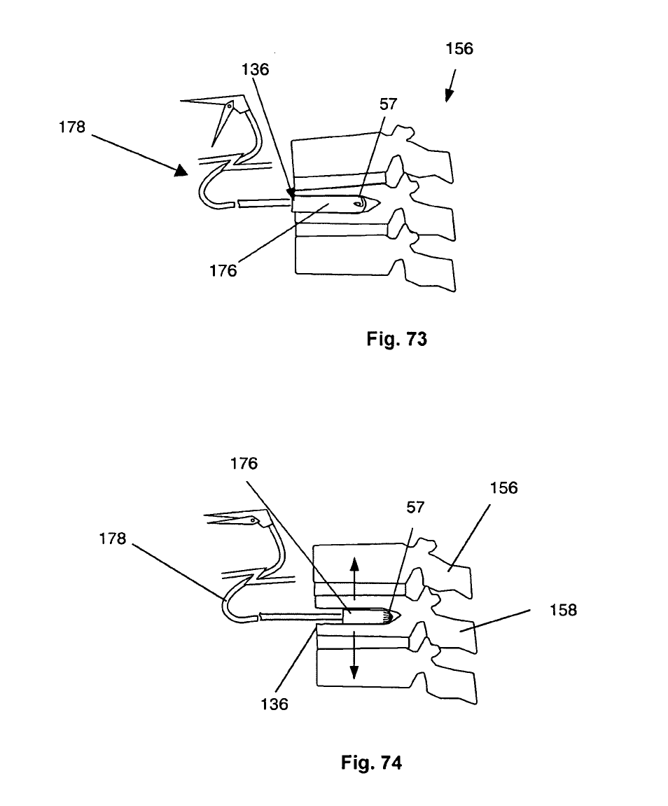

FIGS. 73 and 74 illustrate that a fill cavity 118 of a deployed closable-tip fracture stent 176 can be filled with fill material 74, for example by use of a fill injecting tool 178. The arrows in FIG. 74 illustrate that this action can further expand the closable-tip fracture stent 2, further securing it into the repair site 57.

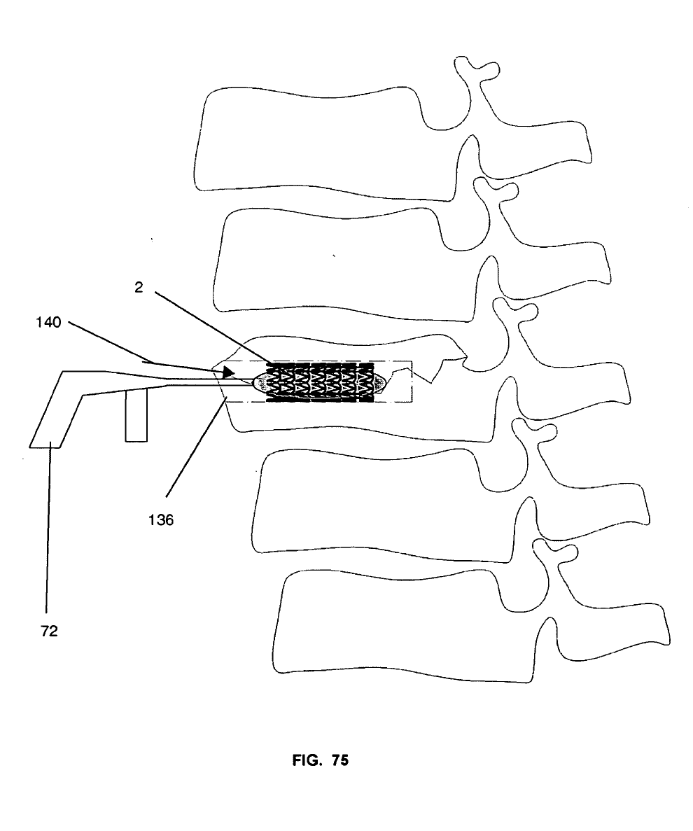

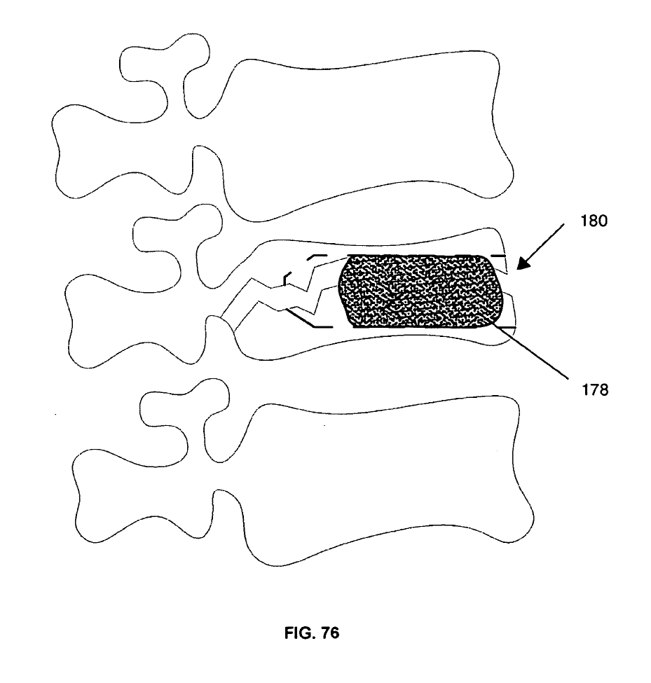

FIGS. 75 and 76 illustrate the injection of a fill material 74 into a closable tip fracture stent 2 deployed in a damage site of a bone 132, for example a fractured vertebra, can help to restore the natural bone structure. FIG. 75 illustrates a closable tip fracture stent 2, for example of the type illustrated in FIG. 34, can be inserted into an access port 136 created in a damage site in a bone 138, for example a compression fracture in a vertebra, by use of a deployment/fill tool 72. FIG. 75 further illustrates that a fill material 74 can be injected, as indicated by the arrow 140, into the closable tip fracture stent 2 by use of the deployment tool/fill tool 72. FIG. 76 illustrates that this injection of fill material 74 into the fracture stent can cause the expansion of the fracture stent 178, thereby restoring the bone to its natural, preinjury, dimension 180.

The closable-tip fracture stent 2 can have a deployed height and a deployed length. The deployed height can be from about 0.3 cm (0.1 in.) to about 5 cm (2 in.), for example about 2 cm (0.6 in.). The deployed length can be from about 0.1 cm (0.05 in) to about 3.8 cm (1.5 in.), for example about 3 cm (1 in.).

The access port 136 can have an access port diameter. The access port diameter can be from about 1.5 mm (0.060 in.) to about 40 mm (2 in.), for example about 8 mm (0.3 in.). The access port diameter can be a result of the size of the access tool. After the closable-tip fracture stent is deployed, the damage site can have a deployed diameter. The deployed diameter can be from about 1.5 mm (0.060 in.) to about 120 mm (4.7 in.), for example about 20 mm (0.8 in.). The deployed diameter can be greater than, equal to, or less than the access port diameter.

U.S. Provisional Patent Application Nos. 60/012,001, filed 21 Sep. 2004; 60/611,972, filed on 21 Sep. 2004; 60/612,723, filed 24 Sep. 2004; 60/612,724, filed 24 Sep. 2004; and 60/612,728, filed 24 Sep. 2004, 60/675,512, filed 27 Apr. 2005; and 60/735,718, filed 11 Nov. 2005 are herein incorporated by reference in their entireties.

It is apparent to one skilled in the art that various changes and modifications can be made to this disclosure, and equivalents employed, without departing from the spirit and scope of the invention. Elements shows with any embodiment are exemplary for the specific embodiment and can be used on other embodiments within this disclosure.

We claim:

1. A biologically implantable device comprising:
a fracture stent having a porous outer wall and defining a hollow cavity within the fracture stent which is in fluid communication with the outside of the fracture stent through the porous outer wall, said fracture stent assuming a first unexpanded configuration and a second, expanded configuration, and an outer sleeve covering at least a portion of said fracture stent.

2. The device of claim 1 wherein the fracture stent has a leading end with a tip configured to seal the leading end with the fracture stent in the second, expanded configuration.

3. The device of claim 1 wherein the fracture stent has a trailing end.

4. The device of claim 3 wherein the trailing end is provided with a hole.

5. The device of claim 4 wherein the hole is a deployment tool hole or fill port.

6. The device of claim 1 wherein the cross-sectional profile of the fracture stent is greater in the second, expanded configuration than in the first, unexpanded configuration.

7. The device of claim 1 wherein the porous outer wall has an array of holes.

8. The device of claim 7 wherein said array of holes contains macroscopic holes.

9. The device of claim 7 wherein said array of holes contains microscopic holes.

10. The device of claim 1 wherein said outer sleeve comprises a wire mesh screen.

11. The device of claim 1 wherein said outer sleeve comprises a thin metal screen.

12. The device of claim 1 wherein said outer sleeve expands as said fracture stent expands from the first configuration to the second configuration.

13. The device of claim 1 wherein the porous wall and outer sleeve are configured to permit a fill material injected into the hollow cavity to leak out of the hollow cavity.

14. The device of claim 1 wherein the porous outer wall comprises struts.

15. A biologically implantable device comprising a hollow body having a semi-rigid wall defining an enclosed cavity, and at least one opening in the wall such that the opening is open to the environment, wherein the body is formed in a substantially uniform elongated shape and has a leading end and a trailing end, the body defining a porous outer wall made of woven interlocking filaments, the body being expandable from a first configuration to a second configuration.

16. A method of treating tissue comprising:
providing a fracture stent having an outer wall defining a hollow cavity within the fracture stent and having an outer sleeve;
inserting the fracture stent into a biological site in a first, unexpanded configuration;
expanding the fracture stent and sleeve into a second, expanded configuration.

17. The method of claim 16 further comprising the step of:
injecting a filler material into the hollow cavity with the fracture stent in the second, expanded configuration such that the filler material leaks out of the fracture stent through the outer wall and outer sleeve.

18. The method of claim 17 wherein the step of injecting a filler further comprises engaging a fill tool with a hole at the trailing end of the fracture stent and injecting the filler material with the fill tool.

19. The method of claim 17 wherein the step of expanding the fracture stent further comprises engaging an insertion tool with a hole at the trailing end of the fracture stent and actuating the insertion tool to expand the fracture stent.

20. The method of claim 17 wherein the step of injecting a filler material includes injecting a filler material comprising a bone material.

21. The method of claim 17 wherein the step of injecting a filler material includes injecting a filler material comprising bone cement.

22. The method of claim 17 wherein the step of injecting a filler material includes injecting a filler material containing bone growth factor.

23. The method of claim 17 wherein the step of injecting a filler material includes injecting a filler material containing a medicinal preparation.

24. The method of claim 23 wherein the step of injecting a filler material containing a medicinal preparation comprises injecting a material containing an antibiotic.
